(19) United States
(12) Patent Application Publication
(10) Pub. No.: US 2005/0084880 A1
(43) Pub. Date: Apr. 21, 2005

(54) Systems and methods for diagnosing & treating psychological and behavioral conditions

(76) Inventors: Ronald Duman, Guilford, CT (US); Samuel Sathyanesan, New Haven, CT (US)

Correspondence Address: FISH & NEAVE IP GROUP;ROPES & GRAY LLP, ONE INTERNATIONAL PLACE, BOSTON, MA, 02110-2624, US

(21) Appl. No.: 10/889,336

(22) Filed: Jul. 12, 2004

Related U.S. Application Data
(60) Provisional application No. 60/486,632, filed on Jul. 11, 2003.

(51) Int. Cl. C12Q 1/68; G06F 19/00; G01N 33/48; G01N 33/50; C12M 1/34

(52) U.S. Cl. 435/6; 702/20; 435/287.2

(57) ABSTRACT

The systems and methods described herein include microarray systems and methods for manufacturing and printing microarrays to provide gene chips capable of detecting signatures of psychiatric conditions, and as well as gene chips and arrays of sequences for such applications. The invention further provides methods of identifying gene signatures for psychiatric conditions, methods of treating such conditions, and methods of identifying therapeutics for the treatment of neurological and psychiatric conditions.

### CROSS-REFERENCE TO RELATED APPLICATIONS

This application claims the benefit of the filing date of U.S. Provisional Application 60/486632, filed Jul. 11, 2003, and entitled “Systems and Methods for Diagnosing & Treating Psychological and Behavioral Conditions”. The entire content of this application is incorporated by reference herein.

### GOVERNMENT SUPPORT

The invention was supported, in whole or in part, by grant number Grant No. MH25642 from the National Institute of Metal Health. The Government may have certain rights in the invention.

### BACKGROUND

The invention described herein relates to novel genes chips capable of detecting gene signatures of psychiatric conditions and related methods.

Psychiatric illnesses effect millions of people each year. These illnesses can wear many masks and have unpredictable courses. The symptoms of mental disorders involve abnormalities of behavior, mood, perception, thinking and intellectual function. Psychiatric disorders have traditionally been classified into two main groups, organic and functional. In organic disorders, the symptoms result from overt brain disease, e.g., dementia, or from metabolic upset or circulatory toxins, e.g., acute delirium, where there is impairment of consciousness, memory disturbance, perceptual disturbance, difficulty in thinking and psychomotor changes.

Functional disorders, such as schizophrenia, affective disorders, and anxiety disorders, constitute the large majority of psychiatric illnesses. In terms of disability and chronicity, schizophrenia is the most serious of all psychiatric illness. It affects 1% of the adult population at some time in their lives. The fundamental abnormality of an affective disorder is a disturbance of mood—either depression or mania. Depression comes in many forms, from seasonal depressions that come and go with the short days of winter to low level chronic depression that lingers for months. There is a feeling of hopelessness, uselessness, loss of usual interest, weight loss, early waking and restlessness. But some people have what are called atypical depressions in which they put on weight and sleep much of the day. Mood disorders have traditionally been conceptualized as neurochemical disorders, but there is now evidence from a variety of sources demonstrating regional reductions in central nervous system (CNS) volume, as well as reductions in the numbers and/or sizes of glia and neurons in discrete brain areas. Although the precise cellular mechanisms underlying these morphometric changes remain to be fully elucidated, the data suggests that mood disorders are associated with impairments of structural plasticity and cellular resilience. Manji H K, Duman R S; Impairments of neuroplasticity and cellular resilience in severe mood disorders: implications for the development of novel therapeutics. Psychopharmacol Bull 2001 Spring; 35(2):5-49.

Of the many psychiatric diseases, depression is one of the most common. Depression is a devastating illness that affects about 17 percent of the population at point one in life, resulting in major social and economic consequences. Significant progress has been made in our ability to treat depression, but not all depressed patients respond to available anti-depressants and therapeutic response requires several weeks or months of treatment. In addition there is still very little understanding of the underlying pathophysiology or treatment of depression.

Thus, there is a need for systems and methods that will provide an increased understanding of the pathophysiology of psychiatric diseases, such as depression, and their treatment.

### SUMMARY OF THE INVENTION

One aspect of the invention provides a gene chip having a plurality of different oligonucleotides attached to a first surface of the solid support and having specificity for genes associated with at least one psychiatric condition, such as depression.

Another aspect of the invention provides a method for determining a gene signature for a psychiatric condition, comprising (i) preparing samples of control and experimental cDNA, wherein the experimental cDNA is generated from a nucleic acid sample isolated from a subject afflicted with the psychiatric condition; (ii) preparing one or more microarrays comprising a plurality of different oligonucleotides attached to a first surface and having specificity for genes associated with the psychiatric condition; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; (vi) identifying a set of genes from the oligonucleotides identified in step (v), thereby determining a gene signature for the psychiatric condition.

Another aspect of the invention provides a method of determining a gene signature indicative of administration of a therapeutic treatment to a subject, the method comprising (i) preparing samples of control and experimental cDNA, wherein the experimental cDNA is generated from a nucleic acid sample isolated from a subject who has received the therapeutic treatment; (ii) preparing one or more microarrays comprising a plurality of different oligonucleotides attached to a first surface, wherein the oligonucleotides are specific to genes; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; (vi) identifying a set of genes from the oligonucleotides identified in step (v), thereby determining a gene signature for the administration of the therapeutic treatment to the subject.

Another aspect of the invention provides a method for predicting the efficacy of a test compound for altering a behavioral response, comprising: (i) obtaining a gene signature representative of the gene expression profile of at least one sample of a selected tissue type from at least one animal subjected to each of at least one of a plurality of selected behavioral therapies which promote the behavioral response; (ii) administering the test compound to at least one test animal; and (iii) comparing gene expression profile data in at least one sample of the selected tissue type from the animal treated with the test compound to determine a degree of similarity with one or more gene signatures; wherein the predicted efficacy of the test compound for altering the behavioral response is correlated to said degree of similarity.

Yet another aspect of the invention provides a method of assessing the efficacy of a treatment in an individual having a psychiatric disorder, comprising (i) determining gene expression profile data in a plurality of patient samples, obtained at multiple time points during treatment of the patient, of a selected tissue type; (ii) determining a degree of similarity between (a) the gene expression profile data in the patient samples; and (b) a gene signature produced by a therapy which has been shown to be efficacious in treatment of the psychiatric disorder; wherein a high degree of similarity is indicative that the treatment is effective.

Another aspect of the invention provides a kit for identifying a compound for treating a behavioral disorder, comprising (i) a database having information stored therein gene signature data representative of the genetic expression response of selected tissue type samples from animals that have been subjected to at least one of a plurality of selected behavioral therapies and wherein the tissue has undergone a desired physiological change; and (ii) a computer program for comparing gene expression profile data obtained from assays wherein a test compound is administered to an animal with the database and providing information representative of a measure of similarity between the gene expression profile data and one or more stored signatures.

The invention also provides a method for conducting a drug discovery business, comprising: (i) generating a database of gene signature data representative of the genetic expression response of a selected neuronal tissue type from an animal that was subjected to at least one of a plurality of behavioral therapies and that has undergone a selected physiological change since commencement of the behavioral therapy; (ii) selecting at least one gene signature and selecting at least one target as a function of the selected gene signatures; (iii) screening a plurality of small molecule test agents in assays to obtain gene expression profile data associated with administration of the agents and comparing the obtained data with the one or more selected gene signatures; (iv) selecting for clinical development test agents that exhibit a desired effect on the target as evidenced by the gene expression profile data; (v) for test agents selected for clinical development, conducting therapeutic profiling of the test compound, or analogs thereof, for efficacy and toxicity in animals; and (vi) selecting at least one test agent that has an acceptable therapeutic and/or toxicity profile.

The invention further provides another method for conducting a drug discovery business, comprising: (i) generating a database of gene signature data representative of the genetic expression response of at least one selected neuronal tissue type from an animal that was subjected to at least one of a plurality of behavioral therapies and that has undergone a selected physiological change since commencement of the behavioral therapy; (ii) administering small molecule test agents to untreated animals to obtain gene expression profile data associated with administration of the agents and comparing the obtained data with the one or more selected gene signatures; (iii)selecting test agents that induce signatures similar to signatures obtainable by administration of behavioral therapy; (iv) conducting therapeutic profiling of the selected test compound(s), or analogs thereof, for efficacy and toxicity in animals; and (v) identifying a pharmaceutical preparation including one or more agents identified in step (v) as having an acceptable therapeutic and/or toxicity profile.

The invention also provides a method for treating a behavioral condition in a patient, comprising identifying a measure of the neural plasticity of a portion of tissue in patient's central nervous system, and administering an agent to the patient in a therapeutic amount sufficient to alter the neural plasticity by a selected amount.

### BRIEF DESCRIPTION OF THE DRAWINGS

The foregoing and other objects and advantages of the invention will be appreciated more fully from the following further description thereof, with reference to the accompanying drawings wherein:

FIG. 1 shows dual color image of the custom growth factor microarray chip PCR products of 645 genes were printed in duplicate on Corning UltraGAPS slides. The glass chip was simultaneously hybridized to cDNA from sham and ECS-treated animals that were indirectly labeled using Cy3 and Cy5 dendrimers, respectively. Four micrograms of hippocampal total RNA was used to generate cDNA for each experiment. The magnified inset shows the housekeeping genes β-actin, cyclophilin, and β-tubulin (yellow box), an upregulated gene, Cox-2 (red box), and a downregulated gene, 3′ untranslated region of cyclin D1 (green box).

FIG. 2 shows the classification of genes upregulated by ECS. Upregulated genes from both acute and chronic ECS-treated rats from two time points (2 and 6 hr) are classified on the basis of known functional roles into the following four categories: (1) growth factor signaling, (2) angiogenesis and vasodilation, (3) neurotransmitter signaling, and (4) transcription factors and kinases. Graphs show the mean ratio (ECS:sham) of fold change in gene upregulation. Dotted horizontal line indicates a ratio of 1 (i.e., no regulation). Error bars represent SEM of four replicates, each from a different animal.

FIG. 3 shows secondary confirmation of microarray data by in situ hybridization and RNA blot assay. Representative photomicrographs of hippocampal sections from ISH and RNA blot assays using radiolabeled riboprobes are shown, and quantified expression from the indicated cell layers is shown by bar graphs on the right. Results are expressed as a percentage of sham and are the mean±SEM of four separate animals, each analyzed in duplicate brain sections. Lanes 1-4 in the RNA blots indicate individual the samples, each from a separate animal, spotted in duplicate. The housekeeping gene cyclophilin was used to normalize the signal from sham and ECS groups. With the exception of NPY, all ISH images are from acute ECS-treated rats. Upregulation of the neuritin gene shown in A(top) is most evident in the DG granule cell layer after either acute or chronic treatments. A significant increase was also observed in the CA1 pyramidal cell layer after chronic ECS. (B) Expression of VEGF in the choroid plexus and induction in the CA1 and CA3 pyramidal cell layers and DG granule cell layer. (C) VGF was significantly upregulated in the DG with acute ECS and in the DG and CA3 pyramidal cell layers with chronic ECS. (D) Regulation of BDNF was confirmed using the RNA blot assay only E, FGF-2 shows prominent expression in the CA2 of both sham and ECS groups with maximal induction seen in the DG.

FIG. 4 shows secondary confirmation of Cox-2, Egr-3, and NPY. Representative photomicrographs of hippocampal sections from ISH and RNA blot assays using radiolabeled riboprobes are shown. Quantified expression from the indicated cell layers is shown by bar graphs on the right. Results are expressed as a percentage of sham and are the mean˜SEM of four separate animals, each analyzed in duplicate brain sections. Lanes 1-4 in the RNA blots indicate individual samples, each from a separate animal spotted in duplicate. A housekeeping gene, cyclophilin, was used to normalize the signal from sham and ECS groups. (A) Cox-2 exhibits low basal expression but is robustly induced in the DG, amygdala, and outer layer of the cerebral cortex (L1). (B) Egr3 is induced exclusively in the DG. (C) NPY, which shows a punctuate pattern of expression in sections from sham-treated animals, showed maximal regulation only with chronic treatment.

FIG. 5 shows analysis of TIMP-1 expression and regulation. A,B, Representative photomicrographs of TIMP-1 after acute (ISH and RNA blot) or chronic ECS (ISH). The results are expressed as a percentage of sham and are the means±SEM of four separate determinations, each representing a different animal. (A) Robust induction of TIMP-1 after acute ECS is seen in the DG, outer layer of the cerebral cortex (L1), and a blood vessel (BV) just below the DG. Note that a significant expression of TIMP-1 in the choroid plexus is shown. (B) After chronic ECS, there is further induction in the L1 region and the BV. Significant expression is seen in the molecular layer (stratum moleculare) of the DG (SM). (C) An enlarged view of the hippocampus is shown. (D) Emulsion autoradiography shows high grain density over cresyl violet-stained cells.

FIG. 6 shows analysis of gene expression and regulation in the choroid plexus. (A) A comparison of growth factor signaling genes expressed in three regions, choroid plexus, hippocampus, and cortex, is shown by a venn diagram, and the 10 most enriched genes in the choroid plexus are listed. The regional expression of midkine (Mdk), IGF2, IGFBP2, cyclophilin, TGFb1, and TGFb3 was measured by RNA blot assay. (B) The bar graph represents signal intensity of corresponding spots. Inset shows the high level of Mdk and IGF2BP expression by ISH. Gene regulation in the choroid plexus in response to chronic ECS was examined from three sets of pooled samples (n=6 animals for each set). (C) (D) Regulated genes were classified into the following two categories: growth factor and angiogenesis signaling (C), and other signaling pathways (D). The results represented as fold change relative to sham. The dotted horizontal line indicates a ratio of 1 or no regulation. E, Secondary confirmation by RNA blot assay is shown for TIMP-1. Error bars represent SEM from three replicates.

FIG. 7 shows immunohistochemical analysis demonstrating ECS regulation of corresponding protein levels for several genes. Immunohistochemistry was performed on sections from chronic ECS or sham animals using fresh frozen cryo cut sections. (A-C), (E), Representative images are shown from chronic ECS (6 hr after ECS) for NPY (A), VEGF (B), neuritin (C), and TIMP-1(E). (D), Cox-2 immunohistochemistry was 2 hr after ECS. (D) Maximal increases in Cox-2 protein were observed in the DG and amygdala and shown with higher power magnification in the inset. (E) TIMP-1 expression in blood vessel (BV) and outer layer of cerebral cortex corresponds to regions of mRNA regulation. Upregulation of NPY, VEGF, and neuritin was most evident in the hilus and is shown with magnified insets. Scale bars: A-C, 25 μm; D, E, 50 μm.

FIGS. 8 and 9 present in table form gene expression profile data representative of profile data from chronic ECS at the 2 hour time point.

### DETAILED DESCRIPTION

1. Overview

The current revolution in molecular genetics offers new hope of identifying genes that may play a role in disease susceptibility, etiology, and diagnosis. Microarray gene analysis has been added to the arsenal of molecular genetic techniques. See M. Schena et al., “Qualitative monitoring of gene expression patterns with a complementary DNA microarray,” Science, vol. 270, pp. 467-470 (1995). Microarray analysis allows an investigator to screen for thousands of genes in a relatively small patient sample such as a single endoscopic biopsy or a small amount of blood (<2 cc). A microarray is a glass slide, microchip, or membrane with cDNA of a plurality, often hundreds or thousands, of known sequences spotted on it. These microarrays then serve as sequence targets for hybridization to cDNA probes prepared from RNA samples from cells or tissues.

Applicants have applied microarray technology to diagnose and treat psychiatric conditions. The invention relates, in part, to sets of genetic markers whose expression patterns correlate with therapeutic treatments of neurological, and in particular, psychiatric conditions. The invention provides not only methods of identifying gene signatures for psychiatric conditions, but also of gene signature of therapies used to treat such conditions. The invention further relates to the application of gene signatures characteristic of therapies to the identification of therapeutic targets, and related business methods and kits.

The systems and methods described herein include microarray systems and methods for manufacturing and printing microarrays to provide gene chips capable of detecting signatures of psychiatric conditions, and as well as gene chips and arrays of sequences for such applications.

The systems and methods described herein provide microarrays that have a plurality of primers immobilized thereon and have specificity for genes associated with psychiatric conditions. In one embodiment the gene chip has a primers specific for BDNF. The gene chip may be employed for microarray analysis of conditions of interest.

Microarray technology, as is generally known in the art, applies a series of probes specific for genes, or genetic material, on a substrate to form an array or matrix. Material from a cell is applied when the cell is in a certain state of interest. Though people often speak of a particular drug's target in the singular, no gene functions in an isolated fashion. Rather, all genes, and the proteins they encode, are parts of larger systems, interacting with each other in complex ways. These systems are known as pathways, and as the expression of a particular gene is regulated by the body to occur at specific times and in specific locations, so the expression of entire pathways of genes is likewise regulated. The aim of expression profiling is to use the patterns of gene expression as a clue for understanding the underlying pathways.

Most expression analyses rely on the mRNA transcripts that are preludes to the synthesis of proteins as the signal that expression has been turned on. cDNA is used as a probe to diagnose the expression of the corresponding gene in a particular tissue or cell type. The cDNA probe has a single strand, enabling it to pair with any mRNA transcripts of the gene that might be present; a successful pairing triggers whatever readout has been engineered into the system. Typically there are redundant probes, enabling expression to be quantified based on the number of mRNA transcripts that bind. By formulating arrays with very large numbers of probes, known as micro-arrays, DNA chips or gene chips, it is possible to measure simultaneously the level of expression of large numbers of genes. More important are differences in the expression profiles of these genes as seen in different tissues and at different stages. A comparison of expression levels using the same probes directed against control (typically healthy) and experiment (typically diseased or treated) tissues, for instance, might reveal that certain genes changed their expression levels during disease while most others stayed the same. One might then infer that these genes have a role in the disease process.

In yet another aspect, the systems and methods described herein relate to a method for treating a behavioral condition, by identifying a physiological characteristic representative of the neural plasticity of a portion of tissue in patient's central nervous system, and administering an agent in a therapeutic amount sufficient to alter the neural plasticity by a selected amount. In another aspect, the invention provides a method for treating a behavioral condition in a patient, comprising identifying a measure of the neural plasticity of a portion of tissue in patient's central nervous system, and administering an agent to the patient in a therapeutic amount sufficient to alter the neural plasticity by a selected amount. As described elsewhere herein, compounds that alter the neural plasticity can be identified using assays described herein. In some embodiments, the behavioral condition is a psychiatric condition.

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof.

II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The terms “microarray,” “GeneChip,” “genome chip,” and “biochip,” as used herein refer to an ordered arrangement of hybridizeable array elements. The array elements are arranged so that there are preferably at least one or more different array elements on a substrate surface, such as paper, nylon or other type of membrane, filter, chip, glass slide, or any other suitable solid support. The hybridization signal from each of the array elements is individually distinguishable.

The terms “complementary” or “complementarity” as used herein refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “compound” and “test compound” refer to any chemical entity, pharmaceutical, drug, and the like that can be used to treat or prevent a disease, illness, conditions, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of cancer. Examples of test compounds include, but are not limited to peptides, polypeptides, synthetic organic molecules, naturally occurring organic molecules, nucleic acid molecules, and combinations thereof.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

As used herein, the term “subject” refers to a cell, tissue, or organism, human or non-human, whether in vivo, ex vivo or in vitro, under observation.

As used herein, the term “increased expression” refers to the level of a gene expression product is made higher and/or the activity of the gene expression product is enhanced. Preferably, the increase is by at least 1.5-fold, more preferably the increase is at least 2-fold, 5-fold, or 10-fold, and most preferably, the increase is at least 20-fold, relative to a control.

As used herein, the term “decreased expression” refers to the level of a gene expression product is made lower and/or the activity of the gene expression product is lowered. Preferably, the decrease is at least 25%, more preferably, the decrease is at least 50%, 60%, 70%, 80%, or 90% and most preferably, the decrease is at least one-fold, relative to a control.

As used herein, the term “gene signature” refers to an experimentally verified subset of values associated with the expression level of a set of gene products from informative genes which allows the identification of a biological condition, an agent and/or its biological mechanism of action, or a physiological process.

As used herein, the term “gene expression profile” refers to the level or amount of gene expression of particular genes, for example, informative genes, as assessed by methods described herein. The gene expression profile can comprise data for one or more informative genes and can be measured at a single time point or over a period of time. For example, the gene expression profile can be determined using a single informative gene, or it can be determined using two or more informative genes, three or more informative genes, five or more informative genes, ten or more informative genes, twenty-five or more informative genes, or fifty or more informative genes. A gene expression profile may include expression levels of genes that are not informative, as well as informative genes. Phenotype classification (e.g., the presence or absence of a psychiatric disorder) can be made by comparing the gene expression profile of the sample with respect to one or more informative genes with one or more gene expression profiles (e.g., in a database). Using the methods described herein, expression of numerous genes can be measured simultaneously. The assessment of numerous genes provides for a more accurate evaluation of the sample because there are more genes that can assist in classifying the sample. A gene expression profile may involve only those genes that are increased in expression in a sample, only those genes that are decreased in expression in a sample, or a combination of genes that are increased and decreased in expression in a sample.

As used herein, the term “informative genes” refers to a gene or genes whose expression correlates with a particular phenotype, such as the presence or absence of a psychiatric condition in an individual or whether a test compound or treatment is predicted to be efficacious. Samples can be classified according to their broad expression profile, or according to the expression levels of particular informative genes. The genes that are relevant for classification are referred to herein as “informative genes.” Not all informative genes for a particular class or phenotype distinction must be assessed in order to classify a sample. Similarly, the set of informative genes that characterize one phenotypic effect may or may not be the same as the set of informative genes for a different phenotypic effect. Typically the accuracy of the classification increases with the number of informative genes that are assessed.

As used herein, the term “neural plasticity” refers to the ability of a cell to make a long term alteration of its circuitry and functionality in response to new inputs, as well as the ability of neural tissue to recover from injury by reorganizing its function to compensate for partial destruction of tissue or loss of function caused by degenerative disorders. Thus, neural plasticity refers to both increased modifiability, in the sense of being able to learn an altered circuitry in response to specific experiences, and also to increase the capacity to repair in the sense of being able to reorganize following various forms of neural damage.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “mood” is used herein to mean an individual's enduring emotional state, while “affect” refers to short-term fluctuations in emotional state. Thus, the term “mood disorder” is used in reference to conditions in which abnormalities of emotional state are the core symptoms. The most common serious mood disorders reportedly seen in general medical practice are major depression (unipolar depression), dysthymic disorder (chronic, milder form of depression), and bipolar disorder (manic-depressive illness).

The term “psychiatric condition” or “psychiatric disorder” is used herein to mean mental, emotional, or behavioral abnormalities. These include but are not limited to bipolar disorder, schizophrenia, schizoaffective disorder, psychosis, depression, stimulant abuse, alcoholism, panic disorder, generalized anxiety disorder, attention deficit disorder, post-traumatic stress disorder, and Parkinson's disease.

The term “bipolar disorder,” as used herein, refers to any of several mood disorders characterized usually by alternating episodes of depression and mania (e.g., bipolar disorder I) or by episodes of depression alternating with mild nonpsychotic excitement or hypomania (e.g., bipolar disorder II). Individual's at risk of developing bipolar disorder include those with a family history of bipolar disorder. Those at greatest risk have first degree relatives which are diagnosed with bipolar disorder I or II.

III. Gene Chips

One aspect of the invention provides gene chips. Gene chips, also called “biochips” or “arrays” or “microarrays” are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and screening of polynucleotide sequences. Microarrays are particularly desirable for their virtues of high sample throughput and low cost for generating profiles and other data.

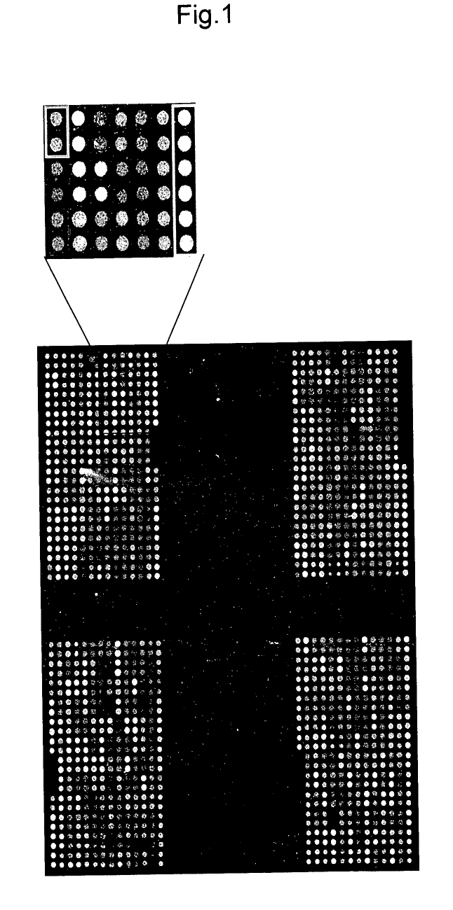

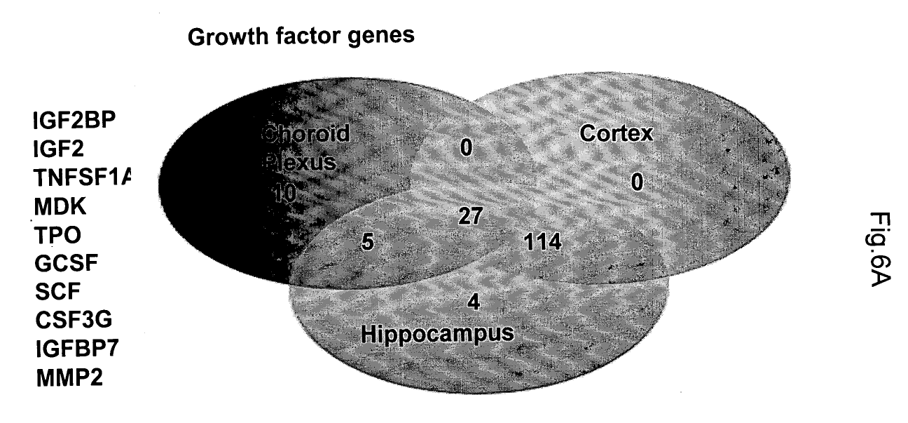

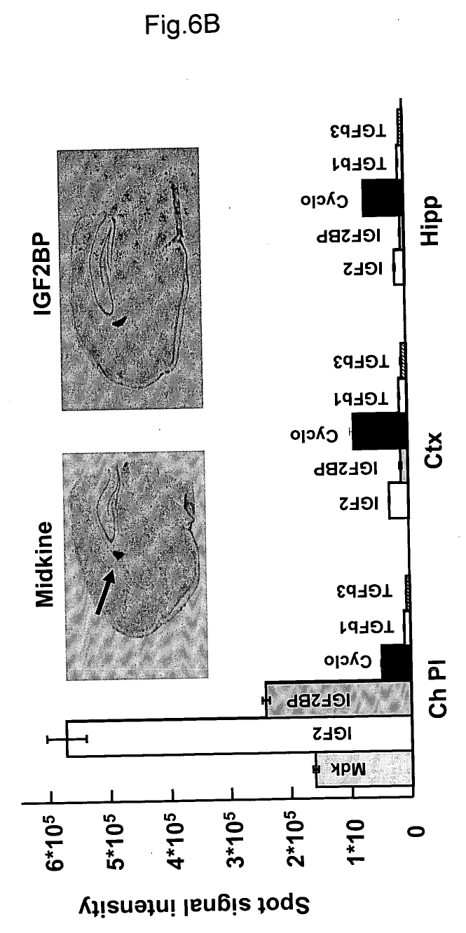

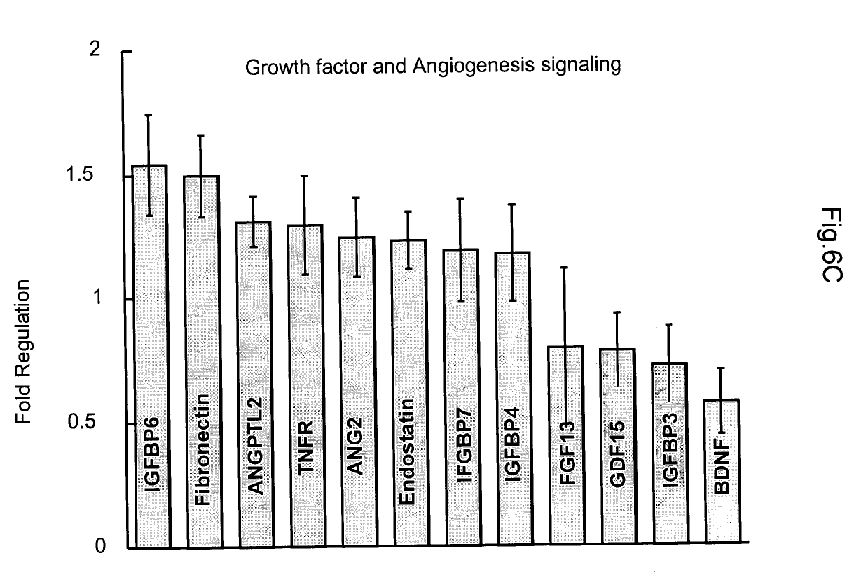

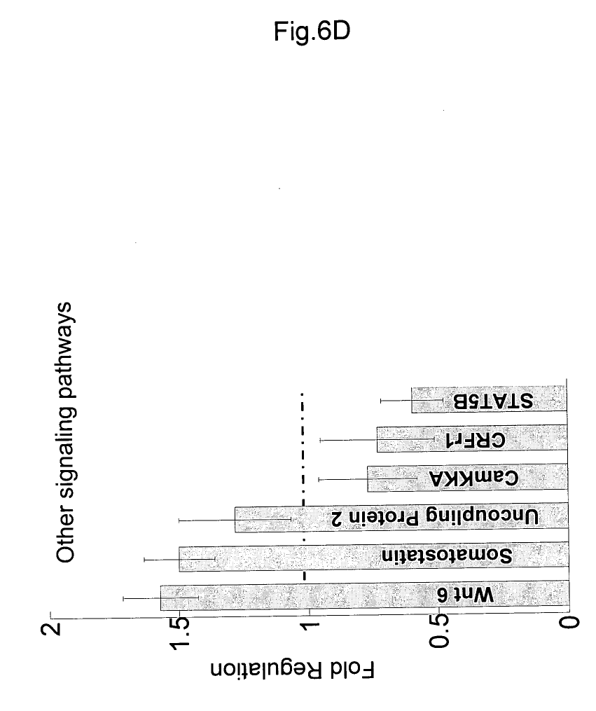

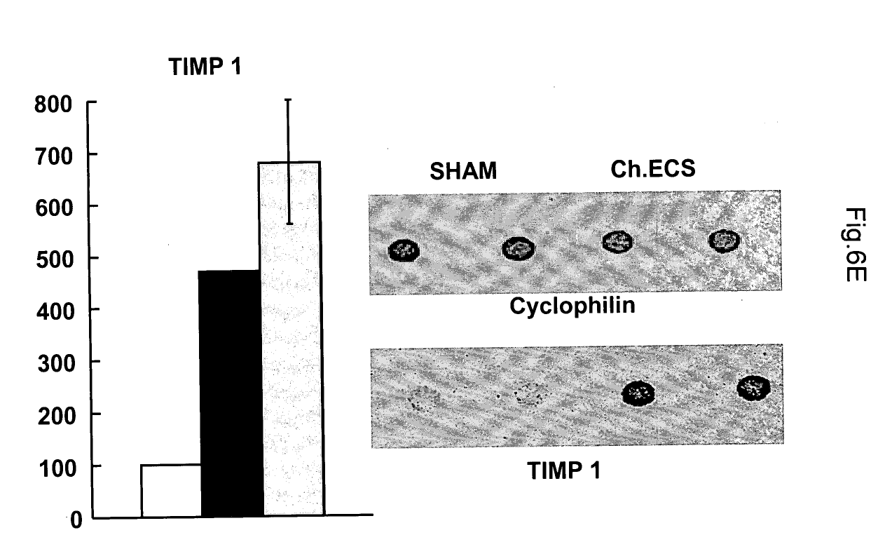

One specific aspect of the invention provides a gene chip having a plurality of different oligonucleotides attached to a first surface of the solid support and having specificity for genes associated with a neurological or a psychiatric condition. In a related embodiment, the invention provides a gene chip having a plurality of different oligonucleotides attached to a first surface of the solid support and having specificity for genes whose expression level changes in a subject who is afflicted with a psychiatric conditions when the subject responds favorably to a therapeutic treatment that is intended to treat the psychiatric condition. In one embodiment of the gene chips provided herein, the oligonucleotides on the gene chip comprise oligonucleotides that are specific for the genes set out in Tables 1-3 or in FIGS. 1 and 6, or in combinations thereof. In another embodiment, the gene chip has oligonucleotides specific for the genes associated with depression. In another specific embodiment, the gene chip has at least one oligonucleotide specific for BDNF. In another specific embodiment, the gene chip has at least one oligonucleotide specific for neuritin, VEGF, VGF, BDNF, FGF-2, Cox 2, Egr3, NPY or TIMP1, or any combination thereof. In another embodiment, the gene chip comprises oligonucleotide probes specific for angiogenic and neurotrophic factors. In one embodiment, the gene chip comprises oligonucleotides specific for ASCL1, HES1 and HES5, or a combination thereof. In another embodiment, the gene chip comprises oligonucleotides specific for BHLHB3, RUNX1, DED, CSDA, HES1, HOXA4, HOXA2, HAND1, BHLHB2, CHX4, EGR3, or combinations thereof. In another embodiment, the gene chip comprises oligonucleotides specific for GIOT1, TEF, CARF, CITED2, ASCL1, JUNDP2, FKHR, HES5 and DBP, or combinations thereof.

In another specific embodiment of the gene chips provided herein, the gene chip comprises at least 3, 5, 10, 15, 20 or 25 of the markers derived from any one of Tables 1-3 or in FIGS. 1 and 6, or combinations thereof. In a related embodiment, at least 50% of the probes on the gene chip are present in any one of Tables 1-3 or in FIGS. 1 and 6. In a related embodiment, at least 70%, 80%, 90%, 95% or 98% of the probes on the gene chip are present in any one of Tables 1-3 or in FIGS. 1 and 6, or combinations thereof.

The invention further provides a gene chip for distinguishing cell samples from individuals having a good prognosis and cell samples from individuals having a poor prognosis, wherein prognosis refers to the prognosis for successful treatment by a given treatment regimen or agent, comprising a positionally-addressable array of polynucleotide probes bound to a support, said polynucleotide probes comprising a plurality of polynucleotide probes of different nucleotide sequences, each of said different nucleotide sequences comprising a sequence complementary and hybridizable to a different, said plurality consisting of at least 5 of the genes corresponding to the markers listed in Tables 1-3 or in FIGS. 1 and 6.

In yet another specific embodiment, the invention provides gene chips that optionally comprise markers additional to at least some of the markers listed in Tables 1-3. For example, in a specific embodiment, the microarray is a screening or scanning array as described in Altschuler et al., International Publication WO 02/18646, published Mar. 7, 2002 and Scherer et al., International Publication WO 02/16650, published Feb. 28, 2002. The scanning and screening arrays comprise regularly-spaced, positionally-addressable probes derived from genomic nucleic acid sequence, both expressed and unexpressed. Such arrays may comprise probes corresponding to a subset of, or all of, the markers listed in Tables 1-3 or in FIGS. 1 and 6, or a subset thereof as described above, and can be used to monitor marker expression in the same way as a microarray containing only markers listed in Tables 1-3 or in FIGS. 1 and 6.

In some embodiments of the gene chips, processes, methods and kits provided by the invention, the psychiatric condition is selected from the group consisting of autism, autism spectrum disorders, Parkinson's disease, parkinsonism, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, Asperger's Disorder, bipolar manic-depressive disorder, ischemia, alcohol addiction, drug addiction, obsessive compulsive disorders, Pick's disease and Binswanger's disease.

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods and gene chips of the invention may be immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or about 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to one aspect of the invention, the microarray is an array (i.e., a matrix) in which each position represents one of the markers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers on Tables 1-3 or in FIGS. 1 and 6 is present on the array.

As noted above, the “probe” to which a particular polynucleotide molecule specifically hybridizes according to the invention contains a complementary polynucleotide sequence. In one embodiment, the probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR: Protocols: A Guide to Methods and Applications, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083). Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the cDNA molecules, and negative control probes, e.g., probes known to not be complementary and hybridizable to sequences in the cDNA molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes may be attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodoret al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller. In one embodiment, the arrays of the present invention are prepared by synthesizing polynucleotide probes on a support. In such an embodiment, polynucleotide probes are attached to the support covalently at either the 3′ or the 5′ end of the polynucleotide.

In a one embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA ARRAYS IN GENETIC ENGINEERING, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm² The polynucleotide probes are attached to the support covalently at either the 3′ or the 5′ end of the polynucleotide.

A First Exemplary Method

In a first exemplary method according to the invention, a neuro-psychiatric gene chip may be prepared through a process wherein a glass slide is prepared as a cDNA microarray. The slide may be a conventional glass slide of the type commercially available from the Corning Company of Corning, N.Y. The slide is preferably GAPS, GAPS II or ULTRA GAPS. More preferably, the amino silane surface on UltraGAPS slides is understood to be more suited to printing long (>50 mer) oligonucleotides. Additionally, the UltraGAPS slide is understood to have a more hydrophobic surface than GAPS II, resulting in smaller spots. However, the invention described herein is not limited to any particular type of slide or substrate, and the substrate selected will turn in part on the printing process employed, as well as the detection process used to identify the presence or absence of a gene of interest.

To contain spot size, the slide is prepared as a microarray having antibodies bound on the chip. In one practice, the antibodies are bound using a micro arrayer, such as the MicroGrid II microarrayer manufactured and sold by the BioRobotics company of Cambridge, UK. The MicroGrid II will handle up to four sample microplates, which can be 96, 384 or 1536 well format. PCR plates can also be used. Up to 48 slides can be printed in a single run. Microarray spotting is carried out with a positional accuracy of around 3 micron, with distance between spot centers being adjustable in 10-micron increments.

In one embodiment, the spot size is controlled by controlling the ambient humidity during the spotting of the probes. In one embodiment, the humidity is kept at between 35%-50%, or more preferably at 40-45%. In another embodiment, the humidity is reduced below 50%, 45%, 40%, 35% or 30%.

As will be discussed in more detail below, a plurality of slides are prepared. A first set of slides are prepared for collecting expression data for control samples and a second set of slides are prepared for collecting expression data for experimental samples. The number of slides prepared may vary according to the application, however it is commonly the case that 3-5 slides are prepared for both the control samples and the experimental samples. This provides sufficient data typically to apply and obtain relevant t-test data to identify statistically significant variations in biological activity.

To prepare the control and experimental samples, the cDNA is synthesized for both control and experiment samples. In one practice, polymerase chain reaction (PCR) is employed to amplify the amount of control and sample material. In each case the amplification process creates strands with different capture sequences.

In one practice of the invention, rather than apply a label to the cDNA sample, a post gene chip application process is employed wherein labeling is applied after sample and experimental material has been hybridized to the gene chips. In this optional practice, there is greater facility to handle low abundance genes. In this process, a new detection system for DNA microarray analysis is employed; it is a simple procedure that does not rely on modified nucleotide incorporation in the fluorescent labeling reaction and allows for high-quality signal detection for high-density DNA microarrays with a high signal-to-background ratio, even over an increasing number of scans of the microarray.

To improve signal detection on cDNA microarrays, we employed a fluorescent oligonucleotide dendrimeric signal amplification system to microarray technology. This signal detection method requires 16-fold less RNA for probe synthesis, does not depend on the incorporation of fluorescent dNTPs into a reverse transcription reaction, generates a high signal-to-background ratio, and can be used to allow for multichannel detection on a single chip. Furthermore, since the dendrimers can be detected individually, it may be possible, by employing dendrimer-binding standards, to calculate or estimate the numbers of bound cDNAs. These features make this dendrimer signal detection reagent attractive for high-throughput functional genomics research.

To this end, dendrimers developed and sold by the Genisphere Company of Hatfield Pa., were employed in the labeling process. These dendrimers are well defined, highly branched macromolecules that emanate from a central core.

Dendritic architecture brings a very high number of functional groups in a compact space. Dendrimers can be tailored to generate uniform or discrete functionalities and possess tunable inner cavities, surface moieties, sizes, molecular weights, and solvent interactions. Dendrimers can be synthesized by a convergent approach, see Tomalia, et al., Macromolecules, 20 at 1164 (1987), alternatively, dendrimers can also be synthesized by a divergent approach, see Tang, et al., Bioconjugate Chem., 7 at 703-714 (1996). As a class, dendrimers are complex, branched molecules built from interconnected natural or synthetic monomeric subunits. A 3DNA dendrimer is constructed from DNA monomers. The use of dendrimers to for the detection microarray hydridization has been described in U.S. Pat. Publication Nos. 2002/0051981 and 2002/0072060, hereby incorporated by reference in their entirety.

Each 3DNA monomer is composed of two DNA strands that share a region of sequence complementarity located in the central portion of each strand. When the two strands anneal to form the monomer the resulting structure has a central double-stranded “waist” bordered by four single-stranded “arms”. This “waist” plus “arms” structure comprises the basic 3DNA monomer.

The single-stranded “arms” at the ends of each of the five monomer types are designed to interact with one another in precise and specific ways. Base-pairing between the “arms” of complementary monomers allows directed assembly of the dendrimer as a step-wise series of monomer layers. The dendrimer monomers bind together in solution to form a dendrimer structure that resembles a ball or cup. Thus, dendrimer complexes of partially double-stranded oligonucleotides are formed, which are stable, spherical structures with a determined number of free ends. Extending from the outer surface of the dendrimer ball or cup are a set of arms that may be tagged.

These arms on the surface of the dendrimer are used to attach the dendrimer's two key functionalities. One function of the arms is to enable attachment of a label. The other is to make the dendrimer specific to the oligonucleotides on the ULTRA GAPS slide.

The molecules that determine the labeling and specificity are attached either as oligonucleotides or as oligonucleotide conjugates. Using simple DNA labeling, hybridization and ligation reactions, the 3DNA dendrimer scaffold may be converted into a highly labeled, application-specific molecule.

The architecture of the 3DNA dendrimer imposes no restrictions on the type of specificity or label used, so the dendrimer may be configured to meet a wide variety of detection needs: fluorescent, enzymatic, or radioactive. The application specificity may take the form of a generic “capture sequence” or it may consist of a target-specific sequence.

In a hybridization reaction, signal intensity is determined by the amount of label that can be localized at the reaction site. The 3DNA dendrimers may be labeled with an average of at least 200 labels. The dendrimer carries this number of labels with it every time it hybridizes to a complementary molecule. The result is up to a 200-fold passive enhancement of signal intensity.

The dendrimer detection method involves first hybridizing the unlabeled first-strand cDNA that contains 5′ dendrimer binding sequence to the microarray, then washing off the excess RT primer and unbound cDNAs. The hybridized cDNAs are then detected by incubating the chips with dendrimers containing the capture sequence on the cDNAs.

Specificity of the dendrimer detection is accomplished through specific binding of capture oligonucleotide on a free arm of the dendrimer. By synthesizing an RT primer consisting of an oligo-dT sequence coupled to a sequence complimentary to the capture sequence on the dendrimer, first-strand cDNA probes are generated, without modified nucleotides, that are capable of binding the dendrimers via the complementary primers. These cDNAs are hybridized to the microarray, and then the unbound material is washed off.

In a second hybridization step, the dendrimers are incubated with the hybridized microarray and allowed to bind to the hybridized cDNAs.

A feature of this detection method is the consistency of fluorescence intensity of the prelabelled dendrimers. Test results show the tightly defined and quantified fluorescent intensity of the dendrimer detection reagent, a feature that should allow for the quantification of the numbers of cDNAs bound to the microarray.

IV. Determining Gene Signatures

One aspect of the invention provides methods for determining a gene signature for a specific neurological disorder or psychiatric condition, such as depression. Furthermore, the systems and methods described herein may be employed to generate gene signatures for diseases of interest. This expression data may be analyzed independently to determine a gene signature of interest, or combined with the existing biological data stored in a plurality of different types of databases. Statistical analyses may be applied as well as machine learning techniques that are used to discover trends and patterns in the underlying data. These techniques include clustering methods, which can be used for example to organize microarray expression data.

One specific aspect of the invention provides a method for determining a gene signature for a psychiatric condition, comprising (i) preparing samples of control and experimental cDNA, wherein the experimental cDNA is generated from a nucleic acid sample isolated from a subject afflicted with the psychiatric condition; (ii) preparing one or more microarrays comprising a plurality of different oligonucleotides attached to a first surface and having specificity for genes associated with the psychiatric condition; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; (vi) identifying a set of genes from the oligonucleotides identified in step (v), thereby determining a gene signature for the psychiatric condition.

In a preferred embodiment, the psychiatric condition is depression. In another embodiment, the psychiatric condition is selected from the group consisting of autism, autism spectrum disorders, Parkinson's disease, parkinsonism, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, Asperger's Disorder, bipolar manic-depressive disorder, ischemia, alcohol addiction, drug addiction, obsessive compulsive disorders, Pick's disease and Binswanger's disease.

In another embodiment, the samples of experimental cDNA may be isolated from a subject or group of subjects afflicted with one or more neurological conditions, one or more psychiatric conditions, or both. Control cDNA may be derived from a nucleic acid sample of a subject or group of subjects which are not afflicted with the neurological or psychiatric conditions that the subjects from which the experimental cDNA was derived. In another embodiment, the subjects from which the experimental and control samples are derived may both be afflicted with the condition, but the severity of the condition or a treatment plan in the two subject groups may differ.

A related aspect of the invention provides a method of determining a gene signature for the administration of a therapeutic treatment to a subject. Such methods are useful to detect the gene expression changes that underlying therapeutic treatments. A gene signature for such genetic changes may be used to determine if a second therapeutic treatment is expected to have the same effect, by comparing the gene expression profile of the second treatment to the gene signature of the first.

Accordingly, one specific aspect of the invention provides a method of determining a gene signature for the administration of a therapeutic treatment to a subject, the method comprising (i) preparing samples of control and experimental cDNA, wherein the experimental cDNA is generated from a nucleic acid sample isolated from a subject who has received or is receiving the therapeutic treatment; (ii) preparing one or more microarrays comprising a plurality of different oligonucleotides attached to a first surface, wherein the oligonucleotides are specific to genes; (iii) applying the prepared samples to the one or more microarrays to allow hybridization between the oligonucleotides and the control and experimental cDNAs; (v) identifying the oligonucleotides on the microarray which display differential hybridization to the experimental cDNA relative to the control cDNA; (vi) identifying a set of genes from the oligonucleotides identified in step (v), thereby determining a gene signature for the administration of the therapeutic treatment to the subject.

In one embodiment of the methods for determining a gene signature for the administration of a therapeutic treatment, administration of therapeutic treatment results in a physiological change in the subject, such as a beneficial change. In a specific embodiment, the physiological change comprises atrophy, growth, loss of neural plasticity, neurogenesis. In another embodiment, the control cDNA may be derived from the subject(s) prior to administration of the therapeutic treatment, or from a subject or group of subjects who do not receive the therapeutic treatment.

In another embodiment of the methods for determining a gene signature for the administration of a therapeutic treatment, therapeutic treatment may comprise a single procedure or it may comprise an aggregate of treatment procedures. In one embodiment, therapeutic treatment comprises a behavioral therapy, such as electroconvulsive seizure therapy, exercise, group therapy, talk therapy, or conditioning. In another embodiment, therapeutic treatment comprises administering to the subject a drug, such as an antidepressant or antipsychotic drug. In another embodiments, the subject is afflicted with a neurological or psychiatric condition. Such condition may be one which the therapeutic treatment is intended to treat. In another embodiment, the subject is a healthy subject who is not afflicted with a neurological or psychiatric condition. In another embodiment, the therapeutic treatment is a treatment for a psychiatric or a neurological condition.

In another embodiment, the drug is a serotonergic antidepressant medication, such as one selected from the group consisting of citalopram, fluoxetine, fluvoxamine, paroxetine, or sertraline, or the drug is a catecholaminergic antidepressant medication, such as bupropion.

In another preferred embodiment of the ongoing methods, both the control cDNA and the experimental cDNA are derived from a nucleic acid sample isolated from the subject. Samples may be isolated from a mammal, such as a human. In a specific embodiment, the sample is isolated post-morten from a human. Nucleic acid samples may be isolated from any tissue or bodily fluid, including blood, saliva, tears, cerebrospinal fluid, pericardial fluid, synovial fluid, aminiotic fluid, semen, bile, ear wax, gastric acid, sweat, urine, or fluid drained from an edema. In some embodiments of the ongoing methods, the sample is isolated from a neuronal tissue or a combination of tissue types, such as hypothalamus, amygdala, pituitary, spinal cord, brainstem, cerebellum, cortex, frontal cortex, hippocampus, choroid plexus, striatum, and thalamus.

In one embodiment of the ongoing methods, the microarray is any one of the microarrays, or gene chips, described herein. In a preferred embodiment, the oligonucleotides on the microarray comprise those listed in Tables 1-3 and/or FIGS. 1 and 6. In a specific embodiment, the oligonucleotides of the microarray are specific to genes encoding growth factors, growth factor receptors, downstream targets of growth factors, genes containing CRE-binding sites, transcriptional regulators, or a combination thereof. In a preferred embodiment, at least 10%, 20%, 30%, 40%, 50%, 60%, 70%, 80%, 90% or 95% of the genes on the microarray are specific to genes selected from Table 1, 2 or 3.

In one embodiment of the ongoing methods, when preparing the microarray, the spot size is controlled by controlling the ambient humidity during the spotting of the oligonucleotides. In one embodiment, the humidity is kept at between 35%-50%, or more preferably at 40-45%. In another embodiment, the humidity is reduced below 50%, 45%, 40%, 35% or 30%.

In another embodiment of the ongoing methods, the control cDNA and the experimental cDNAs are hydridize to the same microarray, while in another embodiment they are hybridized to separate but substantially identical microarrays. If the same microarray is used, the cDNA samples may be labeled using fluorescent compounds having different emission wavelengths such that the signals generated by each cDNA type may be distinguished from a single microarray.

In yet another embodiment of the ongoing methods, the control and experimental cDNA is isolated from two or more subjects. In one embodiment, the control cDNA and experimental cDNA are isolated each from at least 3, 5, 10, 15 or 20 subjects. The cDNAs from each subject may be hybridized to the microarrays separately, or the control cDNAs, or the experimental cDNAs, may be pooled together, such that, for example, an experimental cDNA sample is derived from multiple subjects. In preferred embodiments, the subjects are mammals, such as rodents, primates or humans.

In one embodiment of the ongoing methods, the set of genes in the gene signature comprise genes which have a differential expression in the experimental cDNA relative to the control cDNA. Differential expression may refer to a lower expression level or to a higher expression. In preferred embodiments, the difference in expression level is statistically significant for each gene, or marker, on the set. In preferred embodiments, the difference in expression is at least 10%, 20%, 30%, 40%, 50%, 60%, 70%, 80%, 90%, 100%, 150 or 200% greater in the experimental cDNA than in the control cDNA, or vice versa. A gene signature may comprise all the genes which are differentially expressed between the control and experimental cDNAs or it may comprise a subset of those genes. In some embodiments, the gene signature comprises at least 1%, 2%, 3%, 4%, 5%, 10%, 20%, 30%, 40%, 50%, 60%, 70%, 80%, 90%, 95%, 98% or 100% of the genes having differential expression. Genes showing large, reproducible changes in expression between the two samples are preferred in some embodiments. In preferred embodiments, the gene signature further comprises a subset of values associated with the expression level of each of the genes in the signature, such that gene signature allows the identification of a biological and/or pathological condition, an agent and/or its biological mechanism of action, or a physiological process.

The preparation of samples of control and experimental cDNA may be carried out using techniques known in the art. The cDNA molecules analyzed by the present invention may be from any clinically relevant source. In one embodiment, the cDNA is derived from RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from a sample of cells of the various tissue types of interest, such as the neuronal tissue types, using guanidinium thiocyanate lysis followed by CsCl centrifugation (Chirgwin et al., 1979, Biochemistry 18:5294-5299). n another embodiment, total RNA is extracted using a silica gel-based column, commercially available examples of which include RNeasy (Qiagen, Valencia, Calif.) and StrataPrep (Stratagene, La Jolla, Calif.). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA. CDNA molecules that are poorly expressed in particular cells may be enriched using normalization techniques (Bonaldo et al., 1996, Genome Res. 6:791-806).

The cDNAs may be detectably labeled at one or more nucleotides. Any method known in the art may be used to detectably label the cDNAs. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the cDNAs. Alternatively, random primers may be used in conjunction with PCR methods or T7 promoter-based in vitro transcription methods in order to amplify the cDNAs.

In one embodiment, the detectable label is a luminescent label. For example, fluorescent labels, bioluminescent labels, chemiluminescent labels, and colorimetric labels may be used in the present invention. In one preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In another embodiment, the control and/or experimental cDNAs are detected by using dendrimers as described in the previous section. One embodiment of the ongoing methods wherein dendrimers are used to detect unlabelled cDNA, the methods further comprise scanning the labeled dendrimers to determine an expression level for a gene of interest.

In a further preferred embodiment, the experimental cDNA are labeled differentially from the control cDNA, especially if both the cDNA types are hybridized to the same microarray. The control cDNA can comprise target polynucleotide molecules from normal individuals (i.e., those not afflicted with the psychiatric disorder or subjects who have not undergone to therapeutic treatment). In one preferred embodiment, the control cDNA comprises target polynucleotide molecules pooled from samples from normal individuals. In one embodiment of the methods for generating a gene signature of a therapeutic treatment, the control cDNA is derived from the same subject, but taken at a different time point, such as before, during or after the therapeutic treatment.

Nucleic acid hybridization and wash conditions are chosen so that the cDNA molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the cDNA molecules. Arrays containing single-stranded probe DNA (e.g., synthetic oligodeoxyribonucleic acids) may need to be denatured prior to contacting with the cDNA molecules, e.g., to remove hairpins or dimers which form due to self complementary sequences. Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25.degree. C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B. V.; and Kricka, 1992, NONISOTOPIC DNA PROBE TECHNIQUES, Academic Press, San Diego, Calif. Hybridization conditions may include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

When fluorescently labeled cDNAs or dendrimers are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In one preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

Signals may be recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different psychiatric conditions.

V. Method of Conducting Pharmaceutical Business

Another aspect of the invention provides methods for conducting businesses related to the methods and gene chips provided herein.

One aspect of the invention provides a method for conducting a drug discovery business, comprising: (i) generating a database of gene signature data representative of the genetic expression response of at least one selected neuronal tissue type from an animal that was subjected to at least one of a plurality of behavioral therapies and that has undergone a selected physiological change since commencement of the behavioral therapy; (ii) selecting at least one gene signature and selecting at least one target as a function of the selected gene signatures; (iii) screening a plurality of small molecule test agents in assays to obtain gene expression profile data associated with administration of the agents and comparing the obtained data with the one or more selected gene signatures; (iv) selecting for clinical development test agents that exhibit a desired effect on the target as evidenced by the gene expression profile data; (v) for test agents selected for clinical development, conducting therapeutic profiling of the test compound, or analogs thereof, for efficacy and toxicity in animals; and (vi) selecting at least one test agent that has an acceptable therapeutic and/or toxicity profile.

Another aspect of the invention provides a method for conducting a drug discovery business, comprising: (i) generating a database of gene signature data representative of the genetic expression response of at least one selected neuronal tissue type from an animal that was subjected to at least one of a plurality of behavioral therapies and that has undergone a selected physiological change since commencement of the behavioral therapy; (ii) administering small molecule test agents to test animals to obtain gene expression profile data associated with administration of the agents and comparing the obtained data with the one or more selected gene signatures; (iii) selecting test agents that induce signatures similar to signatures obtainable by administration of behavioral therapy; (iv) conducting therapeutic profiling of the selected test compound(s), or analogs thereof, for efficacy and toxicity in animals; and (v) identifying a pharmaceutical preparation including one or more agents identified in step (v) as having an acceptable therapeutic and/or toxicity profile.

One embodiment of the methods described herein for conducting a drug discovery business method further comprises licensing at least one selected test agent to a manufacturer for manufacture and sale of a pharmaceutical preparation comprising said selected agent. In another embodiment, the behavioral therapy includes electroconvulsive seizure therapy, exercise, group therapy, talk therapy, or conditioning. In another embodiment, the selected physiological change includes atrophy, growth, loss of neural plasticity, neurogenesis, or sprouting of granule cell mossy fiber pathway. In yet another embodiment, prior to administration of behavioral therapy, the animal showed at least one symptom of a psychological abnormality. In a related embodiment, prior to administration of behavioral therapy, the animal was afflicted with at least one neurological or psychiatric condition.

VI. Methods of Identifying or Characterizing Therapeutic Compounds

One aspect of the invention provides methods of identifying, or predicting the efficacy of, test compounds. In particular, the invention provides methods of identifying compounds which mimic the effects of behavioral therapies. In still another aspect, the systems and methods described herein provide a method for predicting efficacy of a test compound for altering a behavioral response, by obtaining a database, e.g., as described in greater detail above, treating a test animal (e.g., a control animal that has not undergone other therapies, such as behavioral therapy) with the test compound, and comparing genetic expression data of tissue samples from the animal treated with the test compound to measure a degree of similarity with one or more gene signatures in said database. In certain embodiments, the untreated animal exhibits a psychological and/or behavioral abnormality possessed by the animals used to generate the database prior to administration of the behavioral therapy.

In another aspect, the systems and methods described herein relate to methods of identifying small molecules useful for treating psychological, psychiatric, and/or neurological conditions. For example, a database of gene signature data representative of the genetic expression response of a selected neuronal tissue type from an animal that was subjected to at least one of a plurality of behavioral therapies and that has undergone a selected physiological change since commencement of the behavioral therapy may be obtained. In an exemplary embodiment, animals (e.g., animals that display a preselected behavioral abnormality, such as depression or schizophrenia) are subjected to behavioral therapy (e.g., electroconvulsive seizure therapy, exercise, group therapy, talk therapy, or conditioning), and their neurological tissues are examined for physiological changes (such as atrophy, growth, loss of neural plasticity, neurogenesis, or sprouting of granule cell mossy fiber pathway, preferably with respect to a particular tissue type, such as hypothalamic, fomix, hippocampal, or other tissue), and genetic expression responses are obtained for tissues that have undergone a desired change. In certain embodiments, the animals are further selected for having undergone a desired change in behavior as well.

From such a database, biological targets can be identified, such as potential therapeutics (e.g., genes that are upregulated and thus may exert a beneficial effect on the physiology and/or behavior of the animal), potential receptor targets (e.g., receptors associated with upregulated proteins, the activation of which receptors may exert a beneficial effect on the physiology and/or behavior of the animal; or receptors associated with downregulated proteins, the inhibition of which may exert a beneficial effect on the physiology and/or behavior of the animal). In certain embodiments, one or more genes, the expression of which differs by a statistically significant amount in a treated animal as compared to an untreated control, may be selected as targets.

Small molecule test agents may then be screened in any of a number of assays to identify those with potential therapeutic applications. The term “small molecule” refers to a compound having a molecular weight less than about 2500 amu, preferably less than about 2000 amu, even more preferably less than about 1500 amu, still more preferably less than about 1000 amu, or most preferably less than about 750 amu. For example, animals or tissue samples may be treated with such test agents to identify those that produce similar changes in expression of the targets, or produce similar gene profiles, as can be obtained by administration of behavioral therapy. Alternatively or additionally, such test agents may be screened against one or more target receptors to identify compounds that agonize or antagonize these receptors, singly or in combination, e.g., so as to reproduce or mimic the effect of behavioral therapy.

Compounds that induce a desired effect on targets, tissue, or animals may then be selected for clinical development, and may be subjected to further testing, e.g., therapeutic profiling, such as testing for efficacy and toxicity in animals. Analogs of selected compounds, e.g., compounds having similar cores but varying substituents and stereochemistry, may similarly be developed and tested. Agents that have acceptable characteristics for therapeutic use in humans or animals may be prepared as pharmaceutical preparations, e.g., with a pharmaceutically acceptable excipient (such as a non-pyrogenic or sterile excipient). Such agents may also be licensed to a manufacturer for development and/or commercialization, e.g., for manufacture and sale of a pharmaceutical preparation comprising said selected agent.

Accordingly, one aspect of the invention provides a method for predicting efficacy of a test compound for altering a behavioral response, comprising: (i) obtaining a gene signature representative of the gene expression profile of at least one sample of a selected tissue type from at least one animal subjected to each of at least one of a plurality of selected behavioral therapies which promote the behavioral response; (ii) administering the test compound to at least one test animal; and (iii) comparing gene expression profile data in at least one sample of the selected tissue type from the animal treated with the test compound to determine a degree of similarity with one or more gene signatures; wherein the predicted efficacy of the test compound for altering the behavioral response is correlated to said degree of similarity.

In one embodiment of the foregoing methods, step (i) comprises obtaining a gene signature representative of the gene expression profile of at least two samples of a selected tissue type. In a related embodiment, step (i) comprises obtaining a gene signature data representative of the gene expression profile of at least three samples of a selected tissue. In one embodiment in which the more than one sample of a selected tissue type is used to determine a gene signature, the selected tissue types are different tissue types, whereas in other embodiments the tissue types are the same. For example, in an exemplary embodiment, a tissue type may be hypothalamus and a second tissue type cortex, such that the gene expression profile data generated from these two tissue samples in the treated animal may be compared to the gene signatures derived from the animal subjected to the behavioral therapy. In other embodiments, gene signatures may be generated from multiple samples of the same tissue type from the same animal, such as blood samples taken at different intervals during the behavioral therapy.

In another embodiment of the foregoing methods, the gene signature is that shown in Table 3. In another embodiment, the gene signature comprises at least 5%, 10%, 20%, 30%, 40%, 50%, 60%, 70%, 80%, 90%, 95% or 98% of the genes shown in Table 3. In another embodiment, the gene signature comprises at least 5, 10, 15, 20, 25 or 30 of the genes listed in Table 3. In another embodiment of the foregoing methods, the gene signature comprises an increase in expression in BHLHB3, RUNX1, DED, CSDA, HES1, HOXA4, HOXA2, HAND1, BHLHB2, CHX4 or EGR3, or a combination thereof. In another embodiment, the gene signature comprises a decrease in expression in GIOT1, TEF, CARF, CITED2, ASCL1, JUNDP2, FKHR, HES5 and DBP, or a combination thereof.

In one embodiment of the foregoing methods, the selected tissue type comprises a neuronal tissue type, such as a neuronal tissue type selected from the group consisting of hypothalamus, amygdala, pituitary, nervous system, brainstem, cerebellum, cortex, frontal cortex, hippocampus, striatum, and thalamus. In another embodiment, the selected tissue type is selected from the group consisting of brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth and tongue.

In one embodiment, the behavioral therapy comprises electroconvulsive seizure therapy, exercise, group therapy, talk therapy, or conditioning. In another embodiment, the behavioral therapy is cognitive-behavioral therapy. Examples of behavioral therapy that may be used in the ongoing methods are described, for example, in Cognitive-Behavioral Therapies by K. Dobson, ed., Guilford Publications, Inc., 2002; The new Handbook of Cognitive Therapy Techniques by Rian E. McMullin; Norton, W. W. & Company, Inc., 2000; and Cognitive Therapy: Basics and Beyond by Judith S. S. Beck, Guilford Publications, Inc., 1995, herein incorporated by reference in their entireties.

In one embodiment of the foregoing methods, the test animal is a human. In another embodiment, the animal is a non-human animal. Such non-human animals include vertebrates such as rodents, non-human primates, ovines, bovines, ruminants, lagomorphs, porcines, caprines, equines, canines, felines, aves, etc. Preferred non-human animals are selected from the order Rodentia, most preferably mice. The term “order Rodentia” refers to rodents (i.e., placental mammals (Class Euthria) which include the family Muridae (rats and mice). In a specific embodiment, the test animal is a mammal, a primate, a rodent, a mouse, a rat, a guinea pig, a rabbit or a human.

The test compound may be administered to the animal using any mode of administration, including, intravenous, subcutaneous, intramuscular, intrastemal, topical, liposome-mediate, rectal, intravaginal, opthalmic, intracranial, intraspinal or intraorbital. The test compound may be administered once or more than once as part of a treatment regimen. In some embodiments, additional test compounds or agents may be administered to the animal to ascertain the efficacy of the test compound or the combination of test compounds or agents. In some embodiments, a gene expression profile may also be obtained from the animal prior to treatment with the test agent. In such embodiments, the efficacy of the test agent may be determined by comparing the gene expression profile of the animal after treatment with the compound with (i) the gene expression profile prior to treatment with the compound and (ii) to the gene signature for the behavioral therapy. For example, if the test compound causes the gene expression profile to approach that of said gene signature, the test compound may be predicted to be efficacious.

It is understood by one skilled in the art that the order of steps (i) and (ii) in the foregoing methods may be interchanged i.e. the animal may be treated with the compound prior to obtaining the genetic data signature for the behavior therapy. Accordingly, the invention also provides a method wherein step (ii) is performed prior to step (i).

When comparing the gene expression profile data in at least one sample of the selected tissue type from the animal treated with the test compound to determine a degree of similarity with one or more gene signatures, any number of statistical methods known to one skilled in the art may be used. In some embodiments, a gene signature may be obtained from samples of a test animal prior to the administration of the test compound or from a control animal to generate a control gene signature for each of the tissue types of interest. In such embodiments, the gene expression profile from the tissue types of the test animal(s) may be compared to both the control gene signatures and the gene signatures resulting from the behavioral therapy to determine to which of these signatures the gene expression profile is most similar. If they are more similar to the control gene signature, the test compound may be considered to less efficacious, whereas if it is more similar to the gene signature of the behavioral therapy then the compound is considered more efficacious.

In one variation of the ongoing methods, more than one test compound may be administered to the test animal, such that the efficacy of a combination of test compounds is tested. In another variation, rather than using, or in addition to using, a test compound, a nonchemical test agent is also applied to the animal, such as temperature, humidity, sunlight exposure or other environmental factor. In yet another environment, the animal is subjected to an invasive or noninvasive surgical procedure, in lieu or in addition to the test compound. In such embodiments, the efficacy of the surgical procedure may be ascertained.

In still yet another aspect, the systems and methods described herein relate to a kit for identifying a compound for treating a behavioral disorder, comprising a database, e.g., as described in greater detail above, and a computer program for comparing gene expression profile data obtained from assays wherein a test compound is administered to an untreated animal with gene expression profile data in the database and identifying similarity between the gene expression profile data from the assays and one or more stored signatures.

Another aspect of the invention provides a method of assessing treatment efficacy in an individual having a psychiatric disorder comprising determining the expression level of one or more informative genes at multiple time points during treatment, wherein a decrease in expression of the one or more informative genes shown to be expressed, or expressed at increased levels as compared with a control, in individuals having a psychiatric disorder or at risk for developing a psychiatric disorder, is indicative that treatment is effective.

The invention also provides a method of assessing the efficacy of a treatment in an individual having a psychiatric disorder, comprising (i) determining gene expression profile data in a plurality of patient samples, obtained at multiple time points during treatment of the patient, of a selected tissue type; (ii) determining a degree of similarity between (a) the gene expression profile data in the patient samples; and (b) a gene signature produced by a therapy which has been shown to be efficacious in treatment of the psychiatric disorder; wherein a high degree of similarity is indicative that the treatment is effective.

Another aspect of the invention provides kits. One aspect provides a kit for identifying a compound for treating a behavioral or psychiatric disorder, comprising (i) a database having information stored therein gene signature data representative of the genetic expression response of selected tissue type samples from animals that have been subjected to at least one of a plurality of selected behavioral therapies and wherein the tissue has undergone a desired physiological change; and (ii) a computer program for (a) comparing gene expression profile data obtained from assays, where a test compound is administered to an animal, with the database; and (b) providing information representative of a measure of similarity between the gene expression profile data and one or more stored signatures.

In some embodiments of the methods described herein, the test compound comprises an antibody, and antisense reagent or a small molecule drug. Test compounds can be screened individually, in combination with one or more other compounds, or as a library of compounds. In one embodiment, test compounds include nucleic acids, peptides, polypeptides, peptidomimetics, RNAi constructs, antisense oligonucleotides, ribozymes, antibodies, and small molecules.

In general, test compounds for modulation of psychiatric disorders can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., Chembridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

Exemplification

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, psychiatric conditions, cognitive therapies or data analysis methods, all without limitation, can be employed, without departing from the scope of the invention as claimed.

The contents of any patents, patent applications, patent publications, or scientific articles referenced anywhere in this application are herein incorporated in their entirety.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

The following methodology was used performing the experiments.

Animals.

Male Sprague Dawley rats (160-180 gm; Charles River Labs, Wilmington, Mass.) were housed, four per cage, under standard illumination parameters (12 hr light/dark cycle) and were given free access to water and food. Animal use procedures were in strict accordance with the National Institutes of Health Guide for the Care and Use of Laboratory Animals and were approved by the Yale University Animal Care and Use Committee.

Custom Growth Factor Chip.

A custom cDNA expression array (645 genes) containing known classes of growth factor families, relevant receptors, cAMP response element (CRE) sequence-containing genes, published antidepressant regulated genes, and well characterized cytokines was produced. The cDNA products spotted on the chip were generated by PCR amplification of rat whole-brain cDNA using stringent amplification conditions. Gene-specific, high-melt temperature PCR primers to the genes of interest were designed using expressed sequence information obtained from public database searches. A Sybr green-based real-time PCR protocol, using the Quantitect Sbyr green PCR mix (Qiagen, Valencia, Calif.), that amplified products with a very high degree of specificity was used for the purpose. Melt-curve analysis was performed on a subset of the genes as another means to ascertain specificity of amplification and purity of the PCR products. PCR products were in the size range of 100-300 bp. The PCR products were precipitated in ethanol-NaCl, washed with cold 70% ethanol, dried, and resuspended in nuclease-free water. Products were printed under denaturing conditions in 50% DMSO buffer onto gamma amino propyl silane or UltraGAPS slides (Coming, Corning, N.Y.) using a robotic microarrayer (GeneMachines; OmniGrid, San Carlos, Calif.).

ECS Treatment.

Rats were divided into the following four groups for each time point (2 or 6 hr): sham acute, sham chronic, acute, and chronic. Rats were placed in plastic cages containing fresh bedding before induction of seizures. Bilateral ECS was administered via moistened pads on spring-loaded ear clip electrodes using a pulse generator (ECT Unit 57800-001; Ugo Basile, Comerio, Italy) (frequency, 100 pulses/sec; pulse width, 0.5 msec; shock duration, 0.5 sec; current, 55 mA) (Madsen et al., 2000b; AC Chen et al., 2001a). This consistently produced a generalized grand mal seizure with characteristic clonic and tonic convulsions. Acute animals received one shock and tissue was collected 2 or 6 hr after shock, whereas chronic animals received 10 shocks over the period of 10 d(one shock each day) and tissue was collected 2 or 6 hr after the last seizure. Sham-treated animals were handled the same as their respective acute and chronic treated animals and had ear clips placed on their ears, but no shock was delivered. All animals were killed by decapitation. The brains were quickly removed and the entire hippocampus was manually dissected and rapidly frozen on dry ice.

Microarray Analysis of Gene Expression.

Total hippocampal RNA from individual animals was isolated using a nonphenolic procedure (RNA Aqueous; Ambion, Austin, Tex.). Optical density values at 260/280 were consistently above 1.9. RNA quality was also determined by reducing gel electrophoresis. Five micrograms of total RNA from ECS-treated and sham-treated rats (n=4) was reverse-transcribed into cDNA and indirectly labeled using a sensitive fluorescent labeling procedure (Genisphere, Hatfield, Pa.). A two-step hybridization and labeling protocol was used where the chip was hybridized to cDNA overnight, washed stringently to remove nonspecifically bound probe, and then poststained with fluorescent dendrimers. After posthybridization washes, slides were scanned using a GenePix scanner (Axon Instruments, Union City, Calif.). Image analysis was performed using GenePix Pro 4.0 software. Resulting files from Genepix 4.0 (Axon Instruments) analysis were imported into Genespring 5.0 (Silicon Genetics, Redwood City, Calif.) for additional visualization and data mining. A gene was considered expressed if its signal intensity was a minimum of twice the background in at least one channel of one-half of the replicates. Per-chip normalization was performed by dividing the expressed genes by the median of two housekeeping control genes that were not regulated, β-tubulin and cyclophilin. Gene regulation was determined by taking the log ratio of the median experimental channel signal to the median control channel signal. Upregulated genes were defined as having an average expression ratio of>1.3, and downregulated genes were defined as having an average expression ratio of<0.7. Raw fluorescence values were obtained directly from the spots for certain genes of interest, including vascular endothelial growth factor (VEGF), VGF, and FGF-2. Secondary validation was performed if they were regulated by≧20% in two of the four samples. Statistical analysis was performed by an unpaired t test using the cross-gene pooled error method in the Genespring software. Genes that had a p value of≦0.05 were then classified into relevant functional categories. The functional classes were further subjected to ANOVA analysis followed by Scheffe's post hoc analysis. This was done for the four treatment groups, acute (2 hr after ECS), acute (6 hr after ECS), chronic (2 hr after ECS), and chronic (6 hr after ECS).

Choroid Plexus Gene Expression.

Rats were administered chronic ECS (10 d) as described above, and the lateral choroid plexus from both hemispheres of sham and ECS was removed by manual dissection and placed in microcentrifuge tubes maintained on dry ice. Animals (n=6) for the choroid plexus experiments were killed 6 hr after the last ECS. The tissue from each experimental group was pooled so that sufficient RNA could be extracted for microarray analysis. RNA extraction, cDNA synthesis, and labeling was accomplished as described above. Experiments were performed using three sets (n=6 for each set) of pooled samples. A fixed ratio was used to determine gene enrichment in the brain regions examined. A gene was considered enriched in a particular brain region if the expression ratio exceeded 1.3. In a comparison of hippocampus versus choroid plexus, genes with a ratio of<0.7 were considered to be enriched in the hippocampus, genes with a ratio between 0.7 and 1.3 were considered to be present equally in the hippocampus and choroid plexus, and genes with a ratio of>1.3 were considered enriched in the choroid plexus.

RNA Blot Assay.

Total RNA was manually spotted in duplicate on Nylon filters (Hybond, positively charged; Amersham Biosciences, Arlington Heights, Ill.) after heating at 80° C. for 10 min. Two microliters of 10-15 ng/˜1 RNA was spotted for each gene of interest, and 5 ng/ul was spotted for housekeeping genes. A fourfold dilution series was included to ensure linearity of signal for each assayed gene. The filters were allowed to dry for 30 min and were cross-linked using a UV cross-linker. Filters were incubated in a prehybridization solution containing 6.25 ml of hybridization buffer (UltraHyb; Ambion), 0.75 ml of formamide, and 14˜1 of ProtectRNase RNase inhibitor (catalog #R7397; Sigma, St. Louis, Mo.) for 1 hr in a rotating oven at 65° C. Radiolabeled riboprobe [same as the probe used for in situ hybridization (ISH), except that ³³P is the radiolabel] of one million counts per milliliter was added to the filters in prehybridization buffer and incubated overnight. Filters were washed twice for 10 min at 65° C. in 2×SSC and 0.1% SDS to remove unbound probe, followed by a 10 min wash at 37° C. in 2×SSC. The following RNase treatment step was included to remove nonspecifically bound probe: 20 min incubation at room temperature in RNase buffer (20 ml of 5 M NaCl, 2 ml of 1 M Tris, pH 8.0, 400 μl of 0.5 M EDTA in 200 ml of distilled water) containing 100 μl of 20 mg/ml RNase A. Filters were then washed in 2×SSC at room temperature for 10 min, followed by a wash at 55° C. in 0.5×SSC and then 0.1% SDS for 10 min. After a final wash in 0.1×SSC at room temperature for 10 min and a rinse in distilled water, filters were placed on glass plates and covered with saran wrap and exposed to a phosphoscreen. Screens were imaged in a phosphorimager (Cyclone; Packard, Meriden, Conn.) and quantified using OptiQuant software.

In Situ Hybridization Analysis.

ISH was performed using radiolabeled riboprobes according to conventional protocols with minor modifications (Newton et al., 2002). Riboprobes were generated by PCR amplification using gene-specific primers. The reverse primer included a T7 template sequence. Whole rat brain cDNA was used as the template for PCR, which was performed in a real-time PCR instrument (SmartCycler, Cepheid, Sunnyvale, Calif.) using the Quantitect Sybr Green PCR kit (Qiagen). PCR product was purified by ethanol precipitation and was resuspended in TE buffer. One microgram of the 300 bp PCR product was used to produce radiolabeled riboprobe using a T7-based in vitro transcription kit (Megashortscript; Ambion). All riboprobes were verified by sequencing of the PCR product. ISH images were quantified using NIH Image software, and statistical analysis was performed using Statview.

Immunohistochemistry.

Immunohistochemical analysis was performed on fresh, frozen, cryocut sections using a protocol that was optimized in our laboratory (Newton et al., 2002). Briefly, cryocut sections were mildly fixed in 4% paraformaldehyde and blocked for 30 min in 2.5% BSA, followed by incubation in primary antibody solution. Unbound antibody was rinsed away before incubation with biotinylated secondary antibody. Nonspecific secondary antibody was removed followed by 1 hr in ABC reagent (Vector Laboratories, Burlingame, Calif.). Antigen detection was done by DAB staining according to the instructions of the manufacturer (Vector Laboratories). Cyclooxygenase-2 (Cox-2) antibody (Cayman Chemicals, Ann Arbor, Mich.) was used at 1:500 dilution, and tissue inhibitor of matrix metalloproteinase (TIMP-1) antibody (R & D Systems, Minneapolis, Minn.) was used at 1:2000 dilution. Sections were dehydrated by a series of alcohol rinses and were coverslipped in DPX (Fluka, Buchs, Switzerland) mountant. Sections were photographed using a digital camera (DVC, Austin, Tex.) under bright-field microscopy using identical exposure times and illumination for sections from sham and ECS animals.

Experiment 1: Gene Signatures of ECS

The role of growth factor signaling in the molecular action of ECS was studied using a customized microarray approach. The glass chip that we developed contains 645 genes, including most known growth factor classes, cognate receptors, known downstream targets, and genes containing CRE sites in their promoter region. The 645 genes were as follows (genes in bold did not yield a PCR product and thus were not included in the gene chip): INHBA, INHBB, INHBC, ADDA, ADDB, ADDG, ANG1, ANG2, ANGPTL1, ANGPTL2, ANGPTL3, ANGPTL4, ANGPTL5, ARTNP, ARTN, BCAT, BCATBP, BSEPC, BAX, BAXEX1/2, BAXA, BAXB, BECL, BCL2MF, BMP1, BMP2, BMP3, BMP4, BMP5, BMP6, BMP7, BMP15, CAMKIA, CAMKIG, CAMKIB1/2, CAMKKB, CAMKKA, CAMKIIA, CAMKIIB, CAMKIID, CAMKIIG, CAMKIV, CASP3, CATNBIP1, CERBB2, CERBB3, CFOS, CJUN, CMOS, CMYCEX1, MIBP1, CNTF, CNTFR, CREB, CSF1M, CSF3G, CSFR1, CSFR2, DPPIV, EGR1, ENDOST, ENOS, ENOSIP, NNOS, EGFR, NSEGFLRD, EGFRRP, EGF, EPO, EPOR, AFGFV1, AFGFV2, AFGFV3, FIBP, FGF4, FGFLF4D, FGF5, FGF6, FGF7, FGF8, FGF9, FGF10, FGF11, FGF12, FGF13, FGF14, FGF14B, FGF15, FGF16, FGF17, FGF18, FGF19, FGF20, FGF21, FGF22, FGF23, FGFBP1, GFG1, GFG2, HBFR2E, HBFR2I, SFGFR3, FRAG1, FIB5, FKHR, FLT3, GAD65, GAD67, GABAARA1, GABAARA2, GABAARA3, GABAARA4, GABAARA5, GABAARA6, GABAARD, GABAARE, GABAARG, GRIF1, GABARAPL2, GABAARRHO1, GABAARRHO2, GABABR1, GABABR2, GABATP, GDNF, GFRA3, GSK3B, GIT1, GCSF, GDF3, GDF9, GDF5, GDF15, VGF, NRG1, HBEGF, HBEGFP, SHBEGF, DIPTX, HGF, HGFR, HDGFRP1, HDGFRP2, IGF2MRNA, IGF2SOMA, IGF2P, IGFR1, IGFR2, IGFBP1, IGFBP2, IGFBP3, IGFBP4, IGFBP5, IGFBP6, IGFBP7, INFA1, INTLK6, INTLK6R, MMP2, MMP9, MAT2A, NGFCDS, NGFA, NGFB, NGFGP, NGFR, NGFRF, TNFRSF16, NGFIA, NGFIB, ORF1, NRTN, NT3, NT4, NT5, NTRK3, NFKB1, PSPN, PDGFA, PDGFAP, PDGFB, PDGFCPOL, PDGFC, PDGFRA, PDGFRAE, PDGFRB, PDGFRBE, 107124, PKA, PKB, PKCI, PKCII, PKCT3, PKCDIII, PKCMU, PKCLAM, PKCTH, PKCDBP, NELL1, NELL2, S100A1HS, S100BHS, S100BRAT, SMAD2, SMAD3, SMAD5, SMAD7, SMADIP1, SMARCD1, SAPKA1, SCF, SSCAM, TPO, TSP1, TIMP1, TIMP2, TIMP3, TIMP4, TGFA, TGFB2P, TGFB2, TGFB2L, TGFMPLS, TGFBPL, TGFBR1, TGFBR3BETAGL, TGFBR3BETAGL, TNFA, TNFSF1A, TNFSF4, TNFSF6, TNFSF11, TNFSF11B, TNFSF8, TYRHYD, JNK1, JNK2, JNK3, MEK1, MEK2, RSK1, MAPKP1, MAPKP2, MAPKP3, PTPPEST, SHPTP1, SHPTP2, CC1, FAK, GRAF, MRDGB, PAP, PRNK, PSGAP, PYK2, P130CAS, PAX, CRAF1, BRAFNTERM, BRAFEX8/9/11, BRAFEX9/10/11, IRS4, P13KP85, PI3KP110, AKT2, AKT3, PTEN, P70S6K, ESTR1, ESTRB2, ESTRB1, CRF, CRFR, CRFBP, CSP, DAD3R, GLUCR, GLYCSYNK3B, HSP70, HSC70, HIP, FNKK, SNKK, CAINTBP, NCAM, NEUPEPY, NPY2R, NPY5R, GAL, NFKB, NETA, NETB, ENK, OPGFR, POMC, 5HTNACT, SUPOXDM1, SUPOXDM3, TETSPCD151, MAOA, MAOB, TRH, TRHRUT, TRHRCDS, TRYPHYDUT, TRYPHYDCDS, EGR3, ZIF268UT, ZIF268CDS, STEP46, STEP61, TNFRCDS, TNFRT2, NTKRT1, VEGF, VEGFD, VEGF1, VEGF3, VEGFR1, VEGFR2, UPAR3, UPAPR, UPAGEN, UPA, WNT1, WNT2, WNT3, WNT4, WNT5A, WNT5B, WNT6, WNT7A, WNT7B, WNT8A, WNTI 1, WNT16, SYNPTOG5, SYNAP1, SYNAPS2, SYNAPS3, NEUR, NEUROF, MINCORTR, E214K, A-CRN, Aphn, Agt, Aqp2, Ahr, Arnt, AANAT, ATF-3, Adrb1, BRCA1, Ctf1, CPTI, CGRP, CGA, Chga, c-Maf, Cox-2, CREM, CRF, Ccna1, CD1-UTR, CD1-CDS, P15INK4b, CFTR, CYTC, Po1B, DABH, BCKDHA, Egr2, Fn1, Fst, FosB, GADD34, Ga1, GIPR, Gcg, Gcr-UTR, Gcr-CDS, GRP78, GST-A3, Hk2, HMGCOA, NOS2, Inha, Ins, I12, I16, JunD, LDHA, SOD2, MITF, MIF, ATP1A1, NSE, NF-1, NF-IL6, NOXA-UTR, NOXA-CDS, NURR1, OCA-B, ODC1, Per1, PEPCK, Pit-1, PPARC1, PENK, PC1, PCNA, PIf, PYC, PDK4, RBA-UTR, RBA-CDS, Amd1a, Scg-2, Sds, Sgk, Sst, Sstr2, Stp-1, Sms, STAT3, TnfA, DI2, Tat, TH, UCP1, UCP2, Avp, VMAT, VIP, IL-1a, IL-1b, IL-1d, IL-1e, IL-2, IL-3, IL-4, IL-5, IL-6, IL-7, IL-9, IL-10, IL-11, IL-12A, IL-12B, IL-13, IL-15, IL-16, IL-17, IL-17b, IL-18, IL-19, IL-20, IL-21, IL-23a, IL1r1, IL1rl1, IL1r2, IL1m, IL2ra, IL2rb, IL2rg, IL3ra, IL4ra, IL5ra, IL6r, IL6st, IL9r, IL10ra, IL10rb, IL11ra1, IL11ra2, IL12rb1, IL12rb2, IL13ra2, IL13ra2, IL15ra, IL17br, IL17r, IL18r1, IL18rap, IL21r, TNF (TNFa), Lta (TNFb), Ltb (Lt-b), TNFR, Tnfsf4 (OX40 ligand), Tnfsf5 (CD40 ligand), Tnfsf6 (FasL), Tnfsf7 (CD27 ligand), Tnfsf8 (CD30 ligand), Tnfsf9 (4-1BB ligand), Tnfsf10 (TRAIL), Tnfsf11 (TRANCE), Tnfsf12 (Apo3L), Tnfsf13 (APRIL), Tnfsf13b, Tnfsf14 (HVEM-L), Vegfb, Vegfc, AIF1, Hgf, Lep, Lif, Ptn, Nfkbia, Casp1, Casp2, Casp3, Casp6, Casp7, Casp8, Casp9, Casp11, Casp12, Casp14, Cyclophilin, ADCYAP1R1, APAF1, ATF2, BCL2, BMP 8, BMP3B (GDF 10), CBLN1, CRH, CRHBP, CRHR1, CRHR2, CSF2 (GM-CSF), CX3CR1, CXCR4, F2, FUS 2, GDNF, GFRA1, GFRA2, GFRA3, GFRA4, GHRH, GMFB, GMFG, GPR74, HCRTR1, HCRTR2, HSPB1, IFNA2, IFNA4, IFNA5, IFNA6, IFNA7, IFNB1, IFNG, IFNW1, MAGED1, MEF2C, MT3, NCOA1, NGFB, NGFR, NGFRAP1, NPFF, NPY1R, NPY6R, NR112, NRG1, NRG2, NTRK1, NTRK2, PENK, PNOC, PPYR1, PSPN, PTGER2, RFRP, RPS6KA6, SNT-1, SNT-2, STAT1, STAT2, STAT3, STAT4, STAT5A, STAT5B, TAC1, TAC3, TNFRSF5, TNFRSF6, TP53, TRO, VGF, Grb 14, Orphanin FR (OFQR), Flk1, BADR-1, NT/N, ACHE, CCK, CHRMGB, GALR1, DIREC, D2REC, DAT, SERT, HTR1A, HTR1B, HTR2A, HTR2C, IGF1, FGFR1, FGFR2, BDNF N, BDNF EX1, BDNF EX2, BDNF EX3, BDNF EX4, BDNF EX5, CEBP A, CEBP B, GBCR5B, SREB1, GPR22, GPR6, GPR26, PER2, PER1, CDKI1, NET1, SUBP, IRS1 PCR, RAT FGF3, IGF1, IGFR1, RAT FGF1, RAT FGF2, ICFBP1, CREBBP, IRS2 PCR, PLCG1PCR, RAT FGF, CARF, BCL2, GPR19, TA1, GPR55, GPR52, SPR, TGFB REC3, FGF REC3, FGF REC4, TGFB REC2, ERK1, ERK3, RDYN, MAP4K, TGFB3, TRKBKIN, RAT ICF2, GPR55, GPR7, GPCRB, RGADPH, HMCCEBP, HMGAPPH, CDK(5), ERF2, CALCNA, CEBP D, CEBP E, CEBP G, MTRKBR2, FGF3, FGFR4, IGF2, IGF2R, FGF4.

The chip exhibited high sensitivity and reproducibility and detected at least 80% of the spotted targets in each hybridization (FIG. 1). Hippocampal gene regulation was examined as a function of either acute or chronic treatments at two time points, 2 and 6 hr after the last seizure. These two time points were chosen so that immediate as well as slow onset genes could be detected In addition, we profiled the genes expressed in the choroid plexus in comparison with the cortex or hippocampus and also examined the regulation of choroid plexus genes in response to ECS. We chose a 30% cutoff value on the basis of the results of previous brain region microarray studies, which show that gene regulation levels in brain tissue are lower than the standard twofold cutoff used for cell culture or other more homogeneous tissues (Mimics et al., 2000). Also, we were able to successfully confirm genes that are regulated by 30%, using independent measures, and frequently observed with in situ hybridization confirmation that the levels of regulation in particular cell layers are much higher than the level of regulation obtained when using total RNA isolated from a dissected brain region.

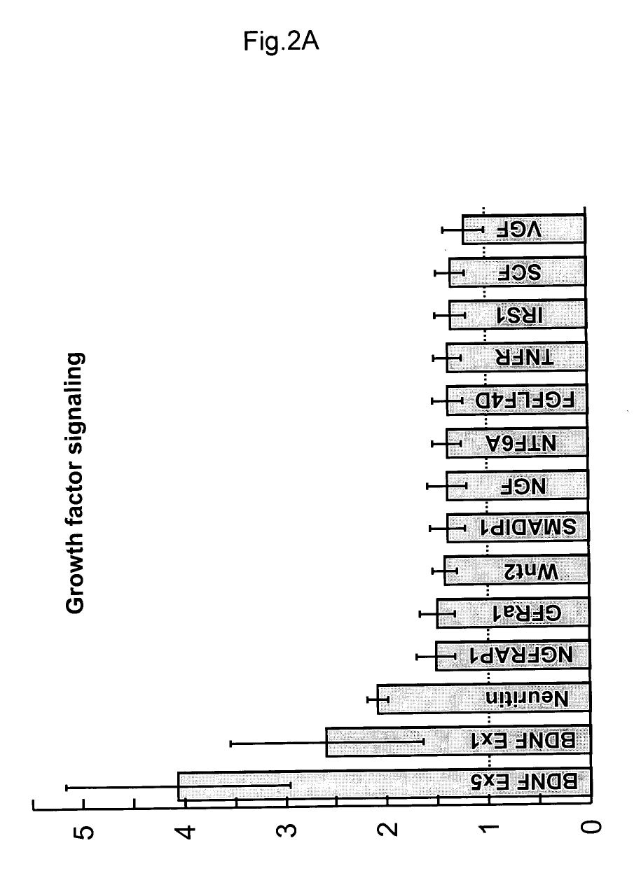

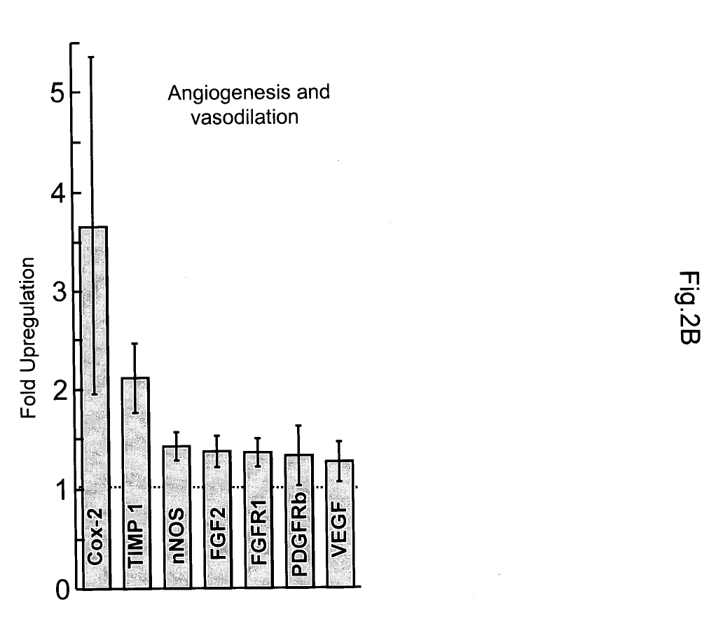

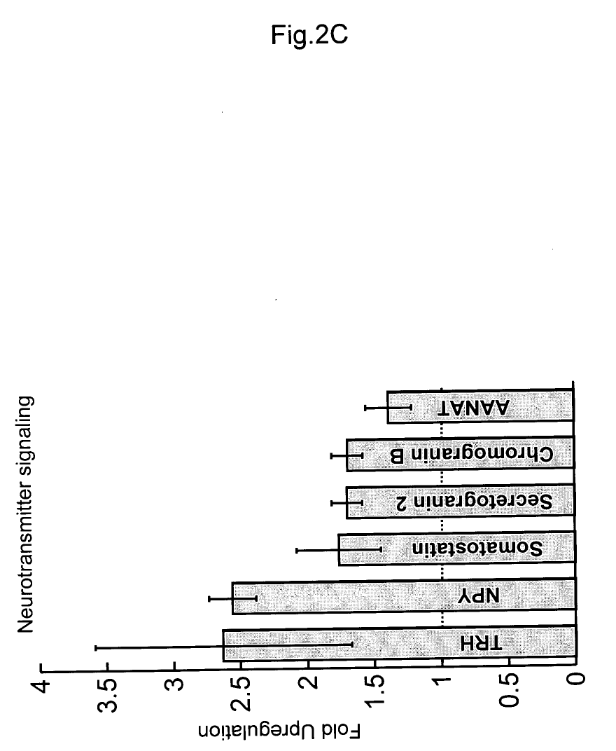

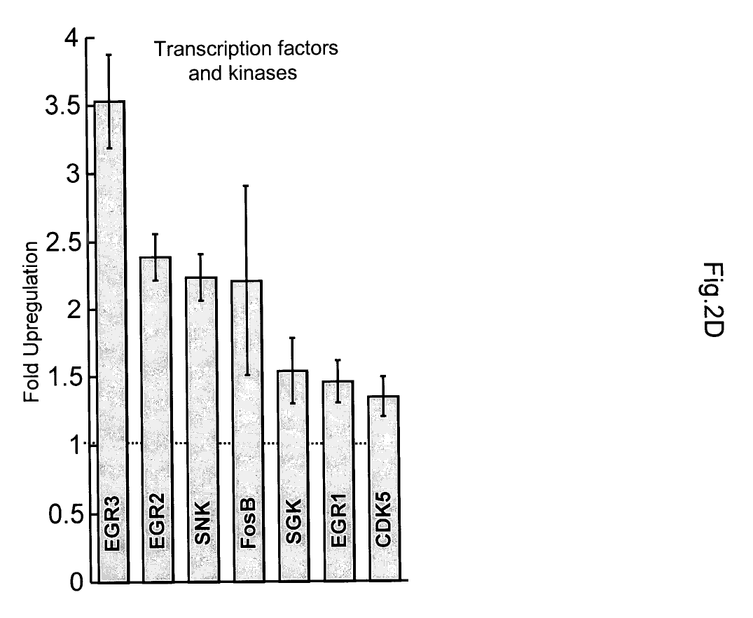

In the hippocampus, 2-7% of the genes were regulated in the various treatment groups, with chronic ECS (6 hr time point) showing the maximum number of regulated genes (37 upregulated, 49 downregulated). The upregulated genes from both acute and chronic ECS at both time points were categorized into four classes on the basis of the following known functional effects: growth factor signaling, angiogenesis and vasodilation, neurotransmitter signaling and transcriptions factors, and kinases (FIG. 2). We chose to include data from acute and chronic ECS from both time points to obtain a wider coverage of regulated genes from our focused array, because our primary intention was to examine the regulation of growth factor signaling after ECS. Most genes that were regulated with acute ECS were also regulated with chronic ECS, except where indicated. Higher levels of regulation but fewer regulated genes were seen with acute versus chronic ECS treatment. Few genes showed regulation in opposite directions when comparing the 2 and 6 hr time points, notably early growth response-2 (Egr-2) and BDNF, which were upregulated at 2 hr for acute and chronic and downregulated at 6 hr.

ANOVA analysis followed by the Scheefe's post hoc analysis for the four functional categories in the four treatment groups, acute (2 and 6 hr after ECS) and chronic (2 and 6 hr after ECS) showed the data to be very significant for several groups (growth factor signaling: acute 2 hr, p<0.009; chronic 2 hr, p<0.0001; angiogenesis and vasodilation: acute 2 hr, p<0.017; acute 4 hr, p<0.04; chronic 2 hr,p<0.0001; neurotransmitter signaling: acute 2 hr, p<0.005; acute 6 hr, p<0.0001; chronic 2 hr, p<0.0006; chronic 6 hr, p<0.0001; transcription factors and kinases: acute 2 hr, p<0.0002). We found several different growth factors to be regulated by ECS. In addition to the factors previously identified (i.e., BDNF and FGF-2), we also found that nerve growth factor (NGF), neuritin, VEGF, and stem cell factor were upregulated. In addition to these factors, several gene products that are involved in growth factor signaling were also upregulated (FIG. 2A). These included the GDNF family receptor 1, Smad interacting protein 1, platelet-derived growth factor receptor, and tumor necrosis factor receptor.

Experiment 2: Secondary Confirmation of Differentially Expressed Genes

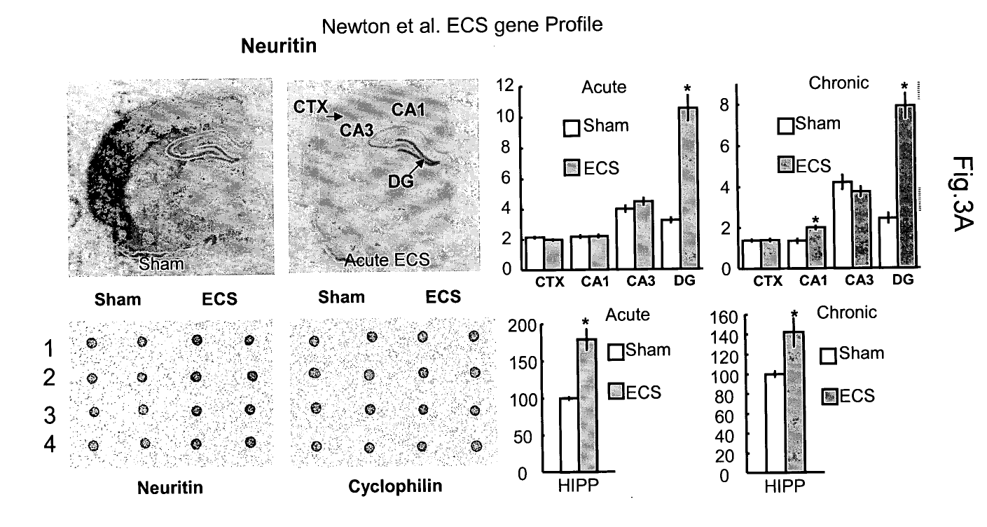

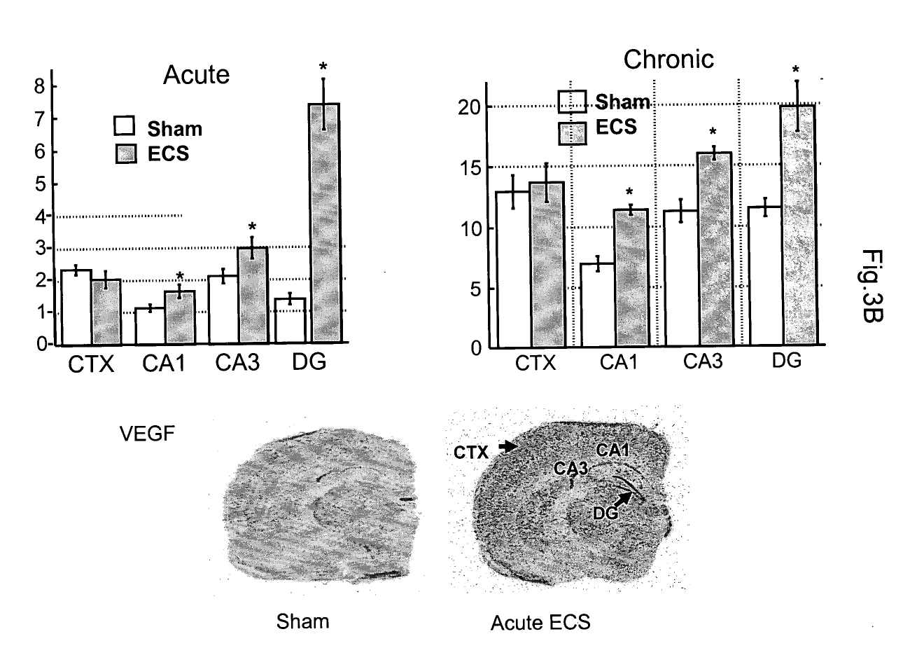

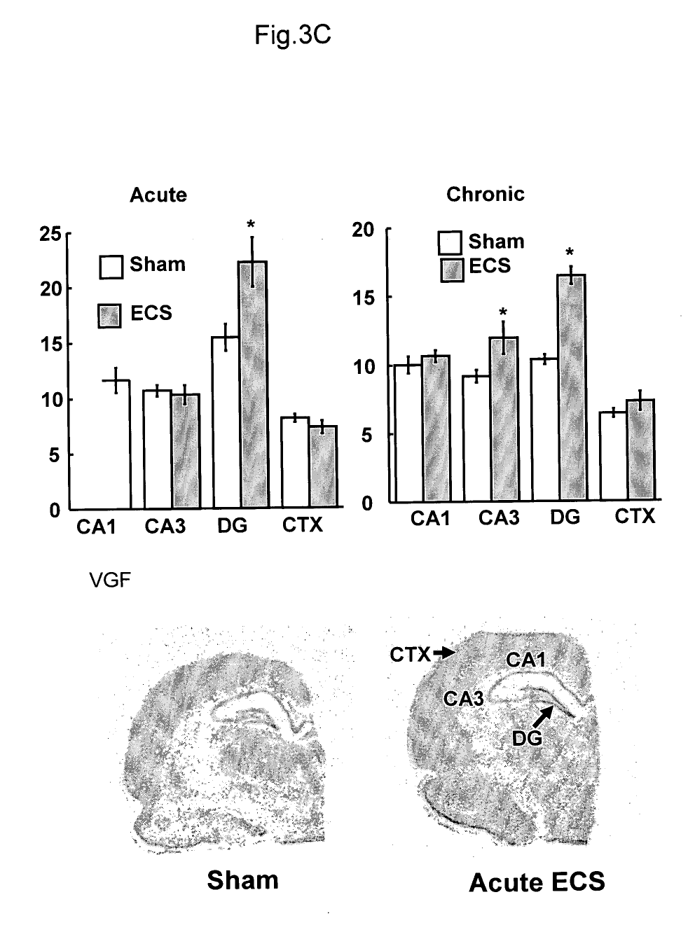

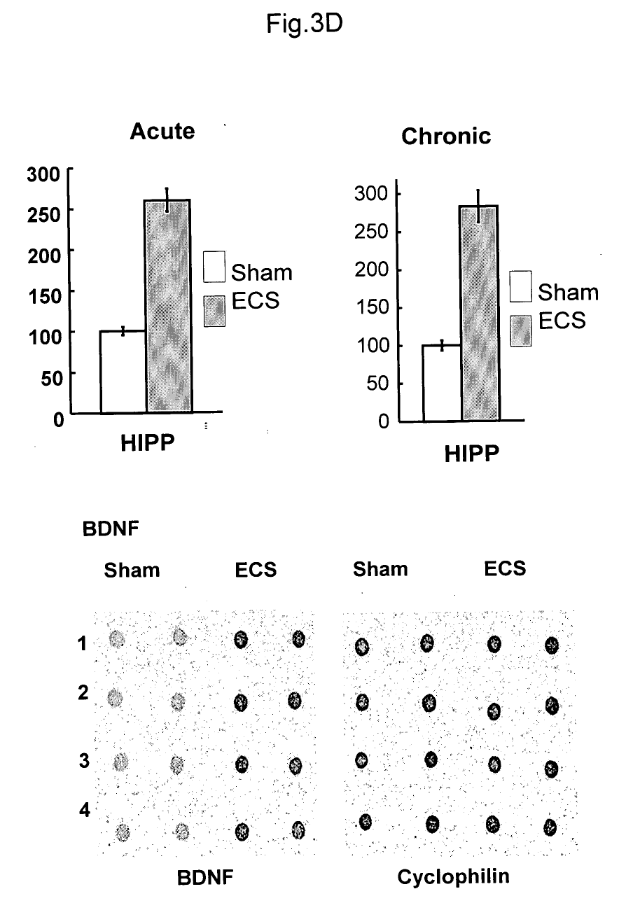

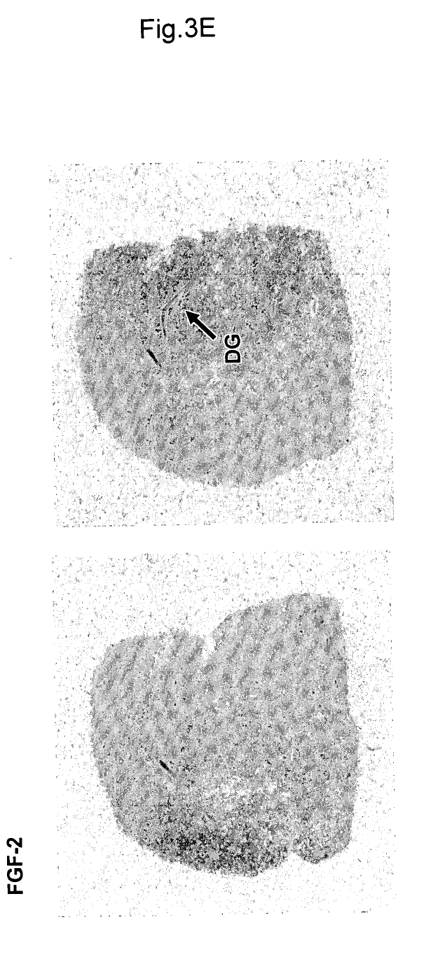

Secondary validation for several regulated genes was performed in an independent set of ECS-treated animals different from those used for the microarray study. Shown in FIG. 3 are representative ISH images for several growth factors or growth factor-induced genes that were identified on the cDNA array. The ISH images are from acute ECS treatments, whereas quantitative graphs are presented for both acute and chronic treatments. Neuritin, an activity and neurotrophic factor-induced gene (Naeve et al., 1997), was upregulated after both acute and chronic ECS (FIG. 3A). The highest level of regulation was in the granule cell layer of the dentate gyrus (DG), although a statistically significant upregulation was seen in the CA1 pyramidal cell layer with chronic ECS treatment. We also optimized a sensitive RNA blot assay, which also confirms the results of the microarray experiments. The RNA blots were conducted using total hippocampal RNA, and there is generally a lower level of regulation than that shown by ISH where cell layer-specific regulation and anatomical distribution data can be obtained.

VEGF and VGF were regulated by only 26 and 21%, respectively (FIG. 2A,B), on the array, but we chose to further examine these two genes because they were consistently regulated across all replicate samples and because they are genes of interest. ISH analysis revealed a much higher level of regulation in specific subregions of the hippocampus, especially the dentate gyrus. VEGF, previously recognized only as an endothelial mitogenic factor, is now emerging as a pleiotropic factor with potent neurotrophic effects (Jin et al., 2002). VEGF was expressed in several brain regions, including the hippocampus and choroid plexus. It is likely that endothelial cells in the brain are also sources of VEGF mRNA. Regulation of VEGF was most pronounced in the DG after acute ECS, but there was also significant induction in the CA1 and CA3 with both acute and chronic ECS (FIG. 3B). This demonstrates neuronal expression as well as regulation of VEGF, lending additional support to the potential neurotrophic actions of this factor. The VGF gene encodes a neuronal and neuroendocrine polypeptide, which is induced by growth factors and neuronal activity (Snyder et al., 1998). This is widely expressed and was induced significantly in the DG (FIG. 3C), almost equally by both acute and chronic ECS. The increase in the CA3 pyramidal cell layer was seen only with chronic treatment. The high level of BDNF induction (FIG. 3D) with ECS has been reported previously (Nibuya et al., 1995).

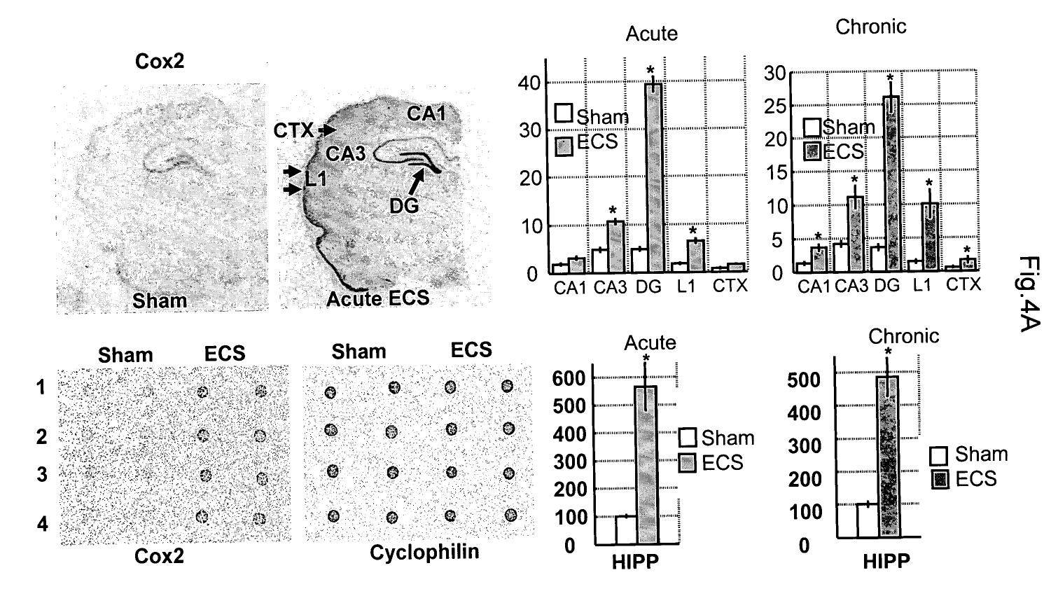

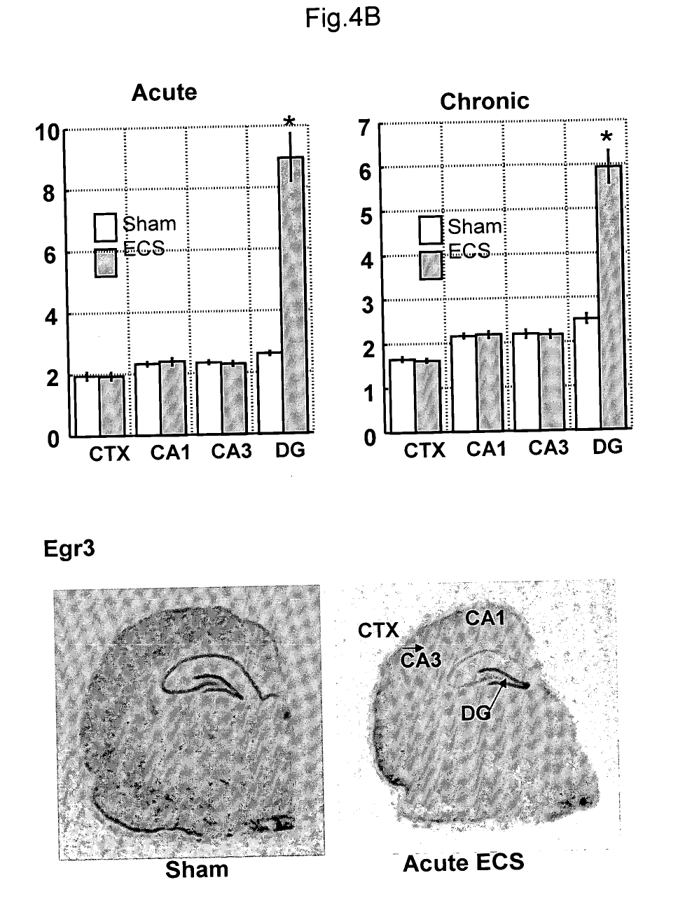

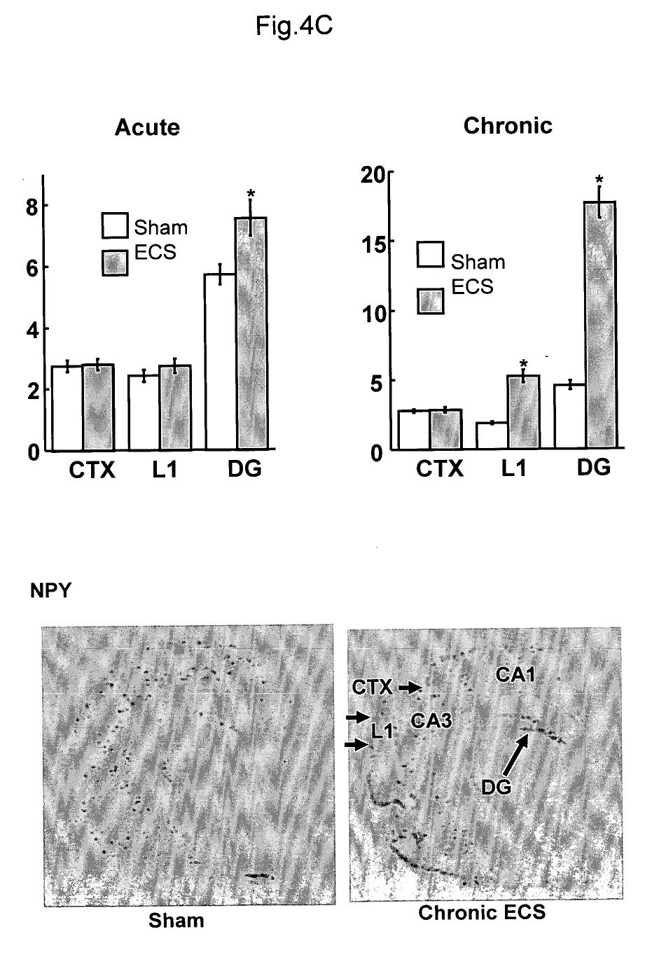

Cox-2, an immediate early gene with low levels of expression in sham animals, is very strongly induced by approximately fivefold by both acute and chronic ECS treatment (FIG. 4A). Cox-2 is upregulated in several forebrain regions, including the CA3 pyramidal and the DG granule cell layers of the hippocampus, the outer layer of the cerebral cortex, and the amygdala. There is a close resemblance in the overall signal intensity from the blots and ISH, with spots from sham animals showing quantifiable but low signal in comparison to signal intensity from treated animals. In contrast, the signal for the housekeeping gene, cyclophilin, is equally high in both sham and ECS-treated rats and not regulated (FIG. 4A, bottom panel). Egr-3, a transcription factor, is upregulated primarily in the DG with little or no regulation in other cell layers of the hippocampus (FIG. 4B). The extent of regulation is similar in both acute and chronic treatments. NPY, a neuropeptide reported to be increased by antidepressant treatment (Husum et al., 2000), shows a punctate expression pattern with a 40% increase in the DG in response to acute ECS and a 3.5-fold increase with chronic ECS (FIG. 4C). There is also an increase in the levels of NPY in the outermost layer of the cortex with chronic ECS.

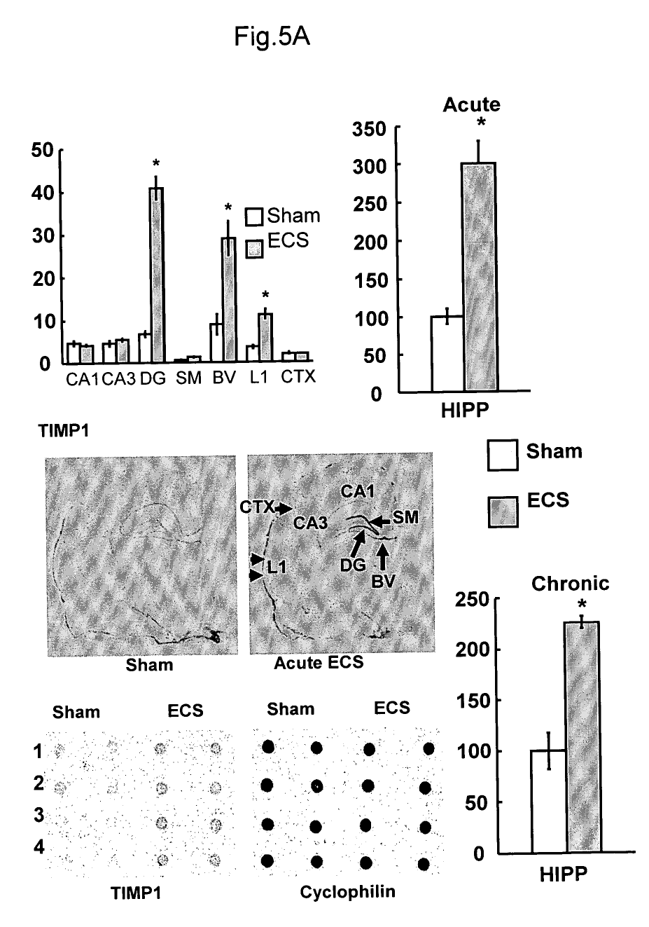

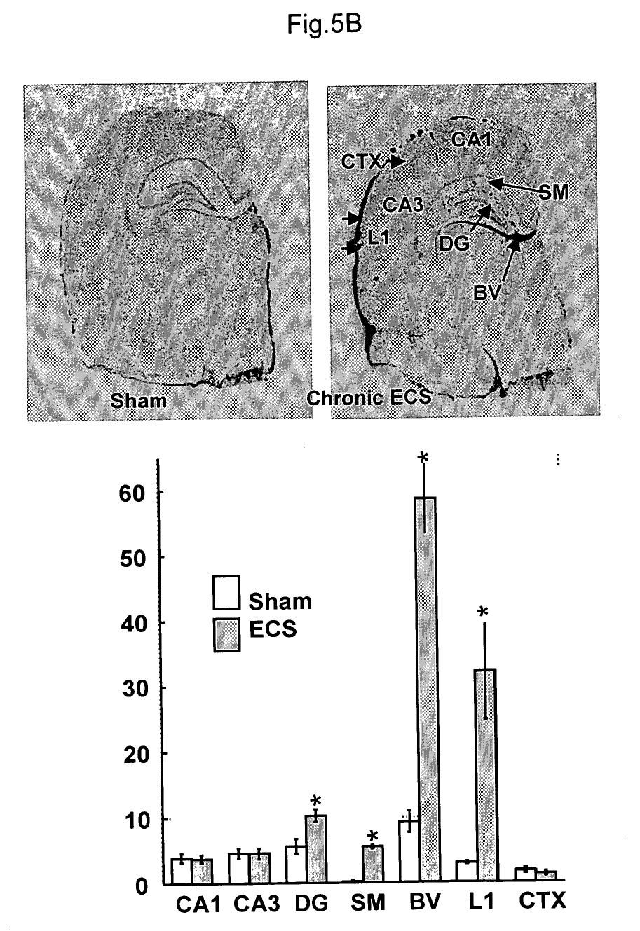

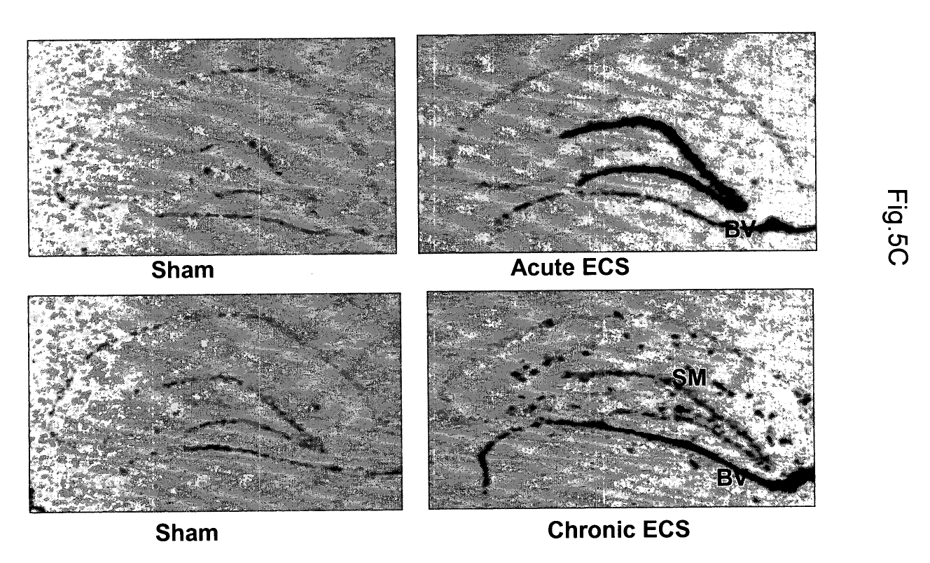

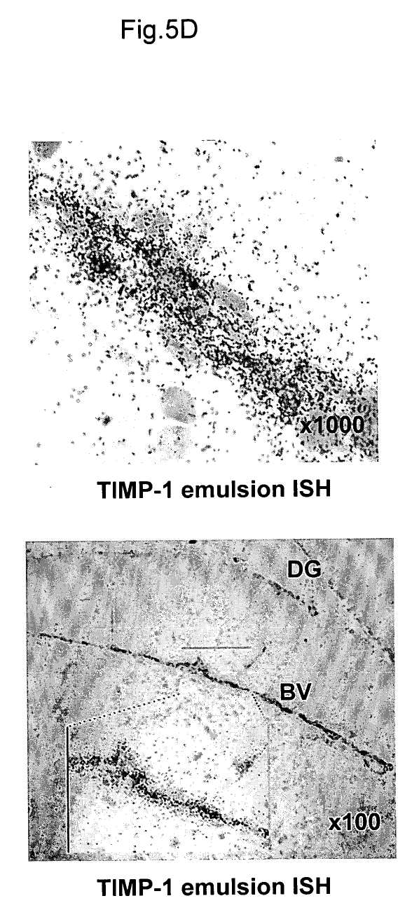

TIMP-1, an inhibitor of matrix metalloproteinases (MMPs), is highly upregulated by approximately threefold in response to either acute or chronic ECS (FIG. 5). MMPs function in the degradation and remodeling of the extracellular matrix (ECM), a necessary event to facilitate new blood vessel sprouting, cell proliferation, and migration. However, it is critical that proteolytic remodeling of the ECM be kept in balance to prevent damage from excessive degradation. This is primarily accomplished by the action of TIMPs. As demonstrated by ISH, the pattern of expression differs after acute (FIG. 5A) or chronic (FIG. 5B) ECS treatment. Basal expression is low and is seen in the outer layer of the cortex, choroid plexus, DG, and CA1 and CA3 pyramidal cell layers of the hippocampus. After acute ECS, strong upregulation (fivefold) is seen in the DG, and a threefold increase is observed in a series of blood elements that run ventral to the hippocampus. There is a 60% increase in the outer layers of the cerebral cortex. Interestingly, after chronic ECS, levels of TEMP-1 in the DG, although significantly increased, are close to basal levels of expression. However, there is an increase in the molecular layer where expression is not seen either in the sham controls or after acute ECS. TIMP-1 expression is further elevated in the blood elements (5.5-fold) and the outer layer of cerebral cortex (fivefold) after chronic ECS (FIG. 5C). To more closely examine the cellular localization of TIMP-1 regulation, ISH sections were dipped in emulsion and developed after 4 weeks. Counter-staining with cresyl violet enabled us to identify TIMP-1 expression in neuronal cells (FIG. 5D).

Experiment 3: Expression of Growth Factor Signaling Genes in the Choroid Plexus

During the secondary confirmation of microarray results by ISH, we noticed high levels of expression of some of the growth factor genes in the choroid plexus. This enrichment of growth factors, combined with the fact that the CP is known to express several growth factor genes and to supply these growth factors to the brain, led us to examine gene expression in the CP relative to the hippocampus and cerebral cortex, as well as gene regulation after chronic ECS (6 hr time point). Of the 171 growth factor genes, 11 were most highly expressed in the hippocampus, and 10 were most highly expressed in the CP. Expression of 13 of the growth factor genes was common to the CP, hippocampus, and cortex (FIG. 6A). A list of genes that had significantly higher expression in CP is also shown. We performed the RNA blot assay to obtain precise levels of expression of some of these genes in the CP relative to the hippocampus and cerebral cortex (FIG. 6B). In addition, ISH for insulin-like growth factor binding protein (IGF2BP) and midkine further demonstrates the high level of enrichment in the CP. The expression of IGF2 and IGFBP2 in the CP is several-fold higher than in either the hippocampus or cerebral cortex. Midkine was below the limits of detection in the hippocampus and the cerebral cortex. Note that the signal for IGF2 and midkine in the CP is several-fold higher than the housekeeping gene cyclophilin, indicating the specialized role of the CP as a repository for these growth factors. This is not the case for the hippocampus or cortex, where cyclophilin is expressed at much higher levels in comparison with other genes of interest (FIG. 6B).

The genes that were regulated in the CP in response to chronic ECS were classified into the following two categories: (1) growth factor and angiogenesis signaling (FIG. 6C), and (2) other signaling genes (FIG. 6D). These two classes were significant using an ANOVA analysis followed by the Scheffe's post hoc test (growth factor and angiogenesis signaling,p<0.01; other signaling pathways, p<0.001). It is rather intriguing to note the strong induction of TIMP-1 (FIG. 6E), which is increased almost fivefold in comparison with the other regulated genes. The regulation of several insulin-like growth factor binding proteins is also noteworthy. The regulation of fibronectin, angiopoeitin 2, and angiopoeitin 2-like protein indicates an angiogenic response, because they are genes with well known roles in angiogenesis (Acker et al., 2001). The upregulation of endostatin, an angiogenesis inhibitor, suggests the presence of a control mechanism in regulating the angiogenic response. With the exception of TIMP-1, which was also confirmed by the RNA blot assay (FIG. 6E), the CP genes exhibit moderate levels of regulation.

Experiment 4: Immunohistochemistry

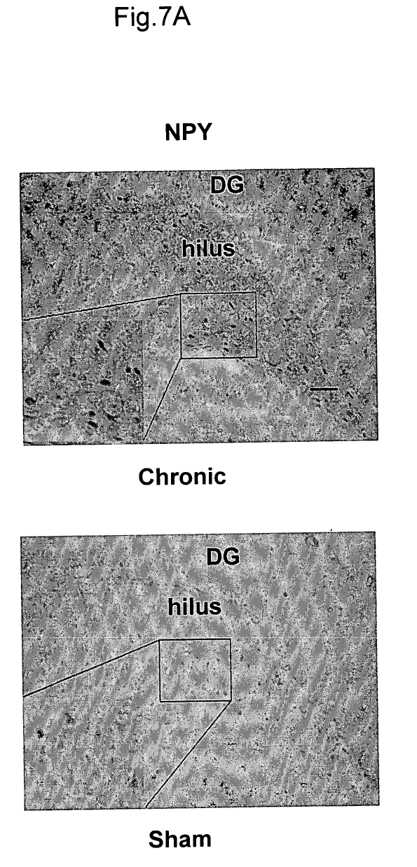

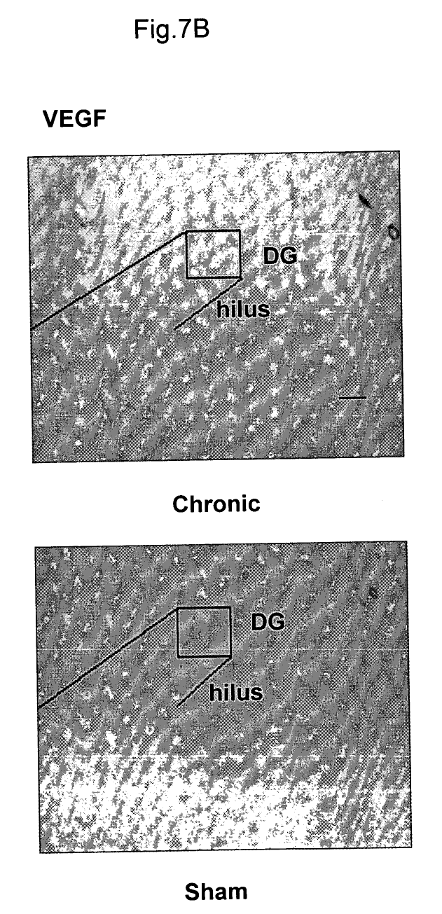

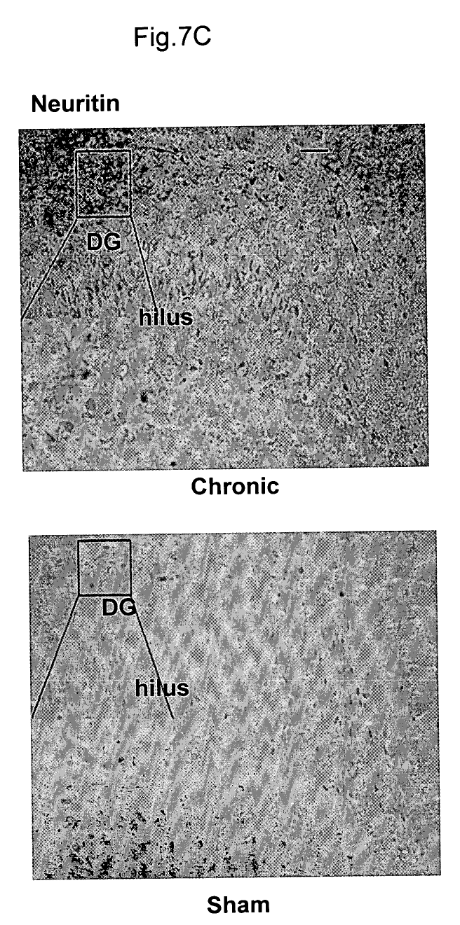

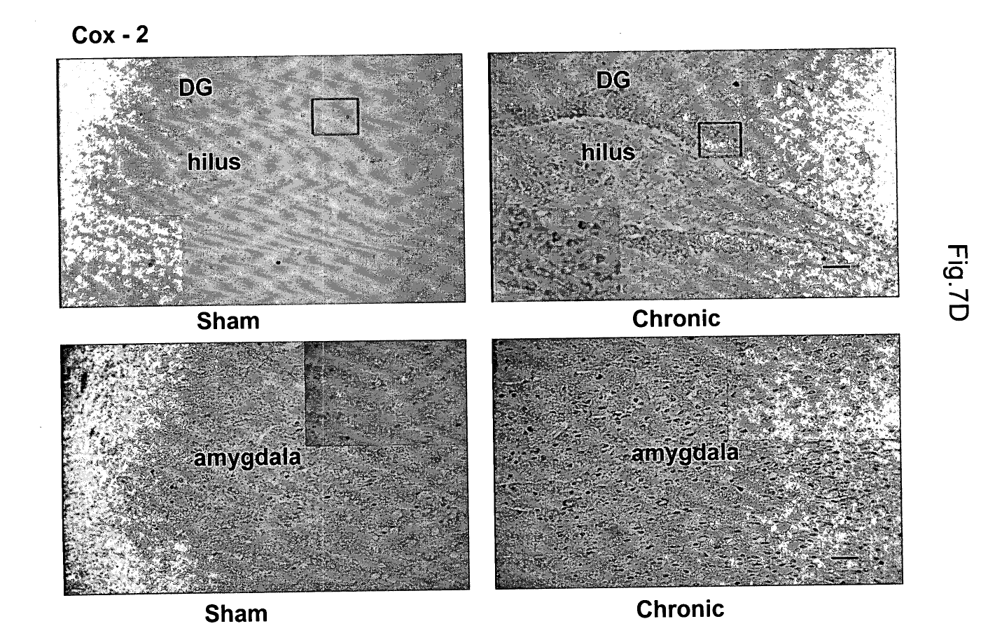

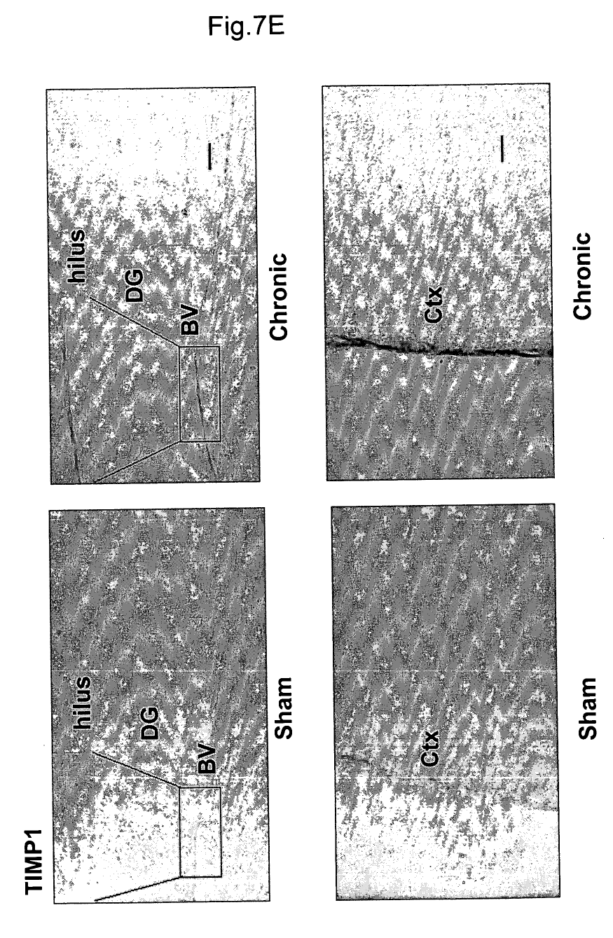

Immunohistochemical analysis was performed on fresh frozen cryocut sections to examine some of the regulated genes at the protein level. With the exception of Cox-2, for which immunohistochemistry was performed at the 2 hr time point, all other molecules examined were from 6 hr after ECS. NPY, which exhibited a punctate mRNA expression, showed protein induction primarily in the hilus (FIG. 7A), an observation that was also seen with VEGF (FIG. 7B) and neuritin immunoreactivity (FIG. 7C). Cox-2 immunoreactivity increased robustly with both acute and chronic ECS in the DG, cerebral cortex (data not shown), and amygdala (FIG. 7D). Basal expression of Cox-2 was very low and almost undetectable, comparable with mRNA expression levels. TIMP-1 immunoreactivity was enhanced in a blood vessel element, in which robust induction of mRNA was seen with chronic ECS (FIG. 7E, top panel). There was also significant induction in the outer layer of the cerebral cortex (FIG. 7E, bottom panel).

Although the above presented exemplary practice uses a post hybridization labeling process, it will be understood that the systems and methods described herein are not so limited and in other embodiments, direct incorporation of fluorescently modified labels may be employed to bind directly to the oligonucleotides.

Additionally, the systems and methods described above may be applied in other applications including, treating mania, anxiety, epilepsy, post traumatic stress disorder and other personality disorders. Additionally, these systems and methods may have application to CNS disorders such as epilepsy and Parkinson disease. Epilepsy is the most prevalent serious neurological condition. About one percent of our country's population suffers from epilepsy. About 60-70% of all epilepsies have no clear cause. Most cases of epilepsy begin before age 20. The hallmark of epilepsy is recurrent electric seizures. About 30% of patients with seizures have an identifiable neurologic or systemic disorder. Epilepsy is an episodic, recurrent, stereotyped phenomenon.

Experiment 5: Gene Signatures for Acute vs. Chronic ECS

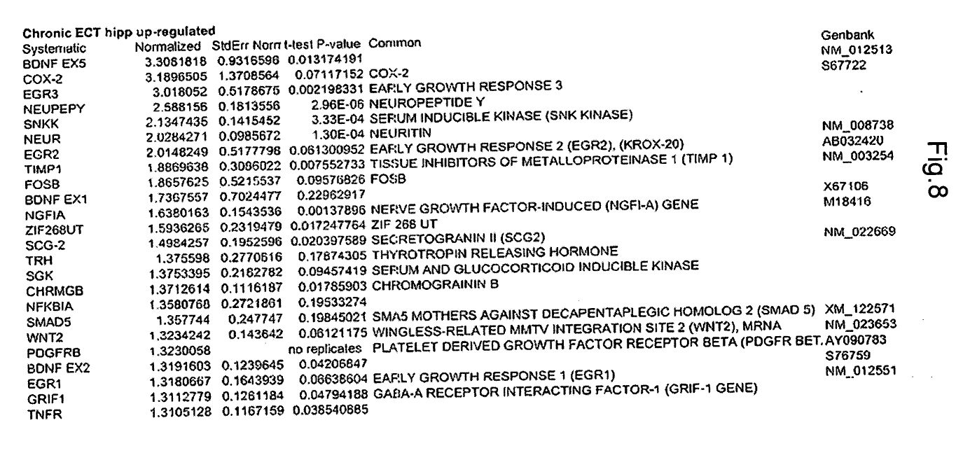

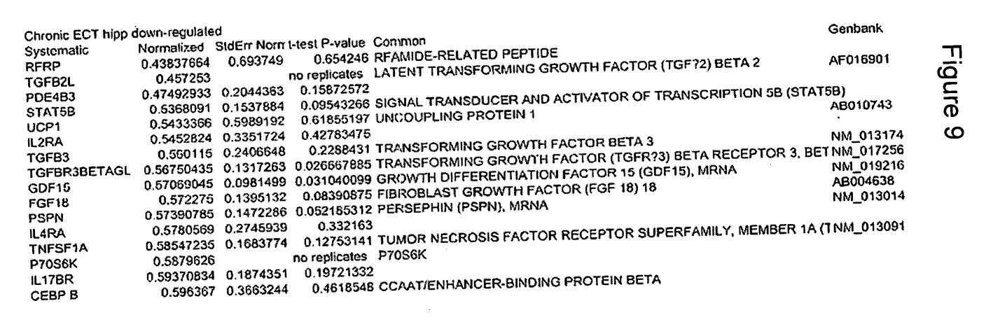

FIGS. 8 and 9 present gene expression profile data from chronic and acute electroconvulsive seizures (ECS) at the 2 hour time point. Experiments were performed as in the preceding section except that a different DNA microarray was used. As shown, the chip contained a plurality of genes including genes that provided a gene signature for chronic ECS (Table 1 below) and genes that provided a signature for acute ECS (Table 2 below):

TABLE 1
Chronic ECS Gene Expression Profile

| Up regulated | Down regulated |
|---|---|
| BDNF-EX5, COX-2, EGR3, | RFRP, TGFB2L, PDE4B3, |
| NEUPEPY, SNKK, NEUR, EGR2, | STAT5B, UCP1, IL2RA, TGFB3, |
| TIMP1, FOSB, BDNF EX1, NGFIA, | TGFBR3BETAGL, GDF15, |
| ZIF268UT, SCG-2, TRH, SGK, | FGF18, PSPN, IL4RA, TNFSF1A, |
| CHRMGB, NFKBIA, SMAD5, | P70S6K, IL17BR, CEBP B |
| WNT2, PDGFRB BDNF EX2, | |
| EGR1, GRIF1, TNFR, | |

TABLE 2
Acute ECS Gene Expression Profile

| Up regulated | Down regulated |
|---|---|
| TNFSF1A, NEUR, | TGFBR1, ESTRB2, IL11RA2, SHPTP2, PDGFC, |
| COX-2, CHRMGB, | TGFMPLS, STAT1, PDE4B2, CD1-UTR, |
| MKD, SNKK, | ESTRB1, PER1, CRFR 1, CGA, VEGFD, |
| IGFBP2, | P70S6K, TGFBR3BETAGL, IL10RA, CEBP A, |
| IGF2MRNA, | SYNPTOGS, TGFA, CRF, TAG1, NT5, FGF14B, |
| BMP5, SCG-2, | NRG1, CARF, IL1R2, TNFRT2, POMC, |
| HSC70, | PDE4D1, RBA-CDS, CAMKIIG, CAMKIB1/2, |
| ENK, CALCNA | FGF16, GPR26, IIL6R, PDGFB, P15INK4B, |
| | IL11 RA1, GDF9, BCATBP, HTR2A, GDF15, |
| | TGFB2L, INTLK6, NT3, TGFB2, STAT5A, |
| | GPR22, MAPKP2 |

ECS involves carefully controlled delivery of electricity into the brain. This electrical stimulation, used in conjunction with anesthesia and muscle relaxant medications, produces a mild generalized seizure or convulsion. While used to treat a variety of psychiatric disorders, it is most effective in the treatment of severe depression, and provides the most rapid relief currently available for this illness. As shown in FIG. 2, ECS appears to increase BDNF mRNA in the chronic case. Moreover, chronic ECS appears to increase the expression of COX-2, EGR3 and NEUPEPY. However, a different gene signature was obtained for acute ECS.

Experiment 6: Gene Signatures for Acute ECS

Table 3 below represents gene expression profile data in the hippocampus from chronic electroconvulsive seizures (ECS) treatment in rat hippocampus. Experiments were performed as in the preceding sections except that a different DNA microarray was used. As shown, the chip contained a plurality of genes including genes that provided a gene signature for chronic ECS (Table 3 below).

TABLE 3
Gene Signatures for Chronic ECS

| Up-regulated | | | | Down-regulated | |
|---|---|---|---|---|---|
| Gene | Normalized | Gene | Normalized | Systematic | Normalized |
| TRH | 90.86948 | ICAM1 | 1.8221694 | IRS1 PCR | 0.70885 |
| TIMP1 | 79.09694 | BHLHB2 | 1.8006728 | NGFRF | 0.705042 |
| TIMP1 R | 45.74955 | UCP2 | 1.7984356 | HTR1A | 0.703687 |
| NPY | 14.84522 | CBLN1 | 1.7922976 | PTN | 0.703589 |
| RUNX1 | 8.920754 | IL18RAP | 1.767147 | ANK3 | 0.703509 |
| ARC | 7.293272 | OXTR | 1.764293 | EML2 | 0.700869 |
| NARP | 6.211001 | OR1A1 | 1.7633896 | SUPOXDM1 | 0.700548 |
| | | BAD_{—} | | | |
| BHLHB3 | 4.98992 | LOC313615 | 1.7571546 | BAD_CDX1 | 0.699625 |
| PTGS2 | 4.864809 | ODC1 | 1.7422187 | MFGE8 | 0.696359 |
| WNT2 | 4.518084 | CTGF | 1.7410891 | CARF | 0.691265 |
| NEUPEPY | 4.402045 | AF313618 | 1.739583 | GPR27 | 0.689924 |
| PENK | 3.822511 | GP38 | 1.7093209 | BAD_TRO | 0.686081 |
| TGFB2P | 3.546189 | MAS1 | 1.7076222 | GPR74 | 0.685988 |
| TNFSF10 | | | | | |
| (TRAIL) | 3.538639 | CSP | 1.6642764 | GIOT1 | 0.674803 |
| GFRA1 | 3.17386 | TH | 1.6581149 | TEF | 0.667576 |
| OCA-B | 3.149084 | OR1N1 | 1.6551319 | 107124 | 0.665433 |
| SCYE1 | 3.118949 | GABASATAA2 | 1.6508005 | SNCB | 0.663261 |
| EGR3 | 2.963339 | TIMP3 | 1.6497285 | SGK | 0.656072 |
| TGFBETA1 | 2.89525 | TOAD 64 | 1.6438233 | CNTNAP1 | 0.655249 |
| SMAD2 | 2.752457 | LOC313815 | 1.6336046 | FIBP | 0.65508 |
| ENK | 2.631728 | VEGF | 1.6267049 | BMYC | 0.652578 |
| CHX4 | 2.574045 | P190RHOGAP | 1.6130677 | GPCR14 | 0.643835 |
| CASP11 | 2.464409 | DVL3 | 1.6130677 | LEPTIN | 0.640106 |
| ADORA3 | 2.422024 | CEBPB | 1.6045105 | BCKDHA | 0.636429 |
| P15INK4B | 2.374172 | VIP | 1.6011193 | ADRA1D | 0.6331 |
| BCAT | 2.373904 | CEBP D | 1.5914367 | GALGT1 | 0.63281 |
| REM2 | 2.359392 | HTR3A | 1.5879128 | FLJ30803 | 0.63208 |
| NF-IL6 | 2.349745 | NR3C2 | 1.5781735 | CAMK1B | 0.627156 |
| FGF2 | 2.185345 | CEBPG | 1.5759859 | CSPG4 | 0.626805 |
| NOGO | 2.181415 | CHRMGB | 1.5672755 | MAAPK3 | 0.625843 |
| DED | 2.169087 | BAD_OR10A1 | 1.5520928 | CAMK1 | 0.621304 |
| BAD_TBX1 | 2.140317 | CRF | 1.5450841 | M1ACHR | 0.612426 |
| BDNF V | 2.140317 | CRF | 1.5450841 | M1ACHR | 0.612426 |
| ULIP | 2.110928 | BAD_GPCRAA | 1.523453 | FKHR | 0.603016 |
| AIF1 | 2.099698 | NCOA1 | 1.5071594 | OR7A17 | 0.60271 |
| RAT FGF2 | 2.059979 | MAOB | 1.4964815 | EDG1 | 0.595407 |
| PDGFB | 2.045925 | GIT1 | 1.4945004 | ILK | 0.594669 |
| VGF | 2.017468 | S100A4 | 1.4894893 | CITED2 | 0.593553 |
| CSDA | 2.002456 | CAMKIIB | 1.4790097 | ASCL1 | 0.586764 |
| GABAARA4 | 1.989376 | NFKBIA | 1.4648098 | JUNDP2 | 0.576442 |
| NEUR | 1.979965 | VTN | 1.4579136 | CTCF | 0.576096 |
| HES1 | 1.974846 | GTF2B | 1.4366829 | LRRN3 | 0.565883 |
| GRB2 | 1.945456 | IL11RA1 | 1.4356669 | DBP | 0.538043 |
| HOXA4 | 1.943954 | GCSF | 1.4338381 | HES5 | 0.518927 |
| F3 | 1.917926 | CCR4 | 1.4312593 | GRB 14 | 0.515109 |
| HAND1 | 1.90965 | FCGRT | 1.428994 | GPR12 | 0.514035 |
| PAR1 | 1.894133 | NT/N | 1.4228327 | G22P1 | 0.514035 |
| GPR85 | 1.892488 | UGFBP7 | 1.4212806 | SMADIP1 | 0.513646 |
| DE4D1 | 1.876551 | HOXA2 | 1.4208032 | GABAARG | 0.512879 |
| GPR18RAT | 1.859992 | ELK1 M | 1.4180237 | CXCL12 | 0.452383 |
| CREM | 1.839216 | OR3A2 | 1.4135681 | CHRD | 0.343781 |
| P2RY12 | 1.826765 | CRAF1 | 1.4039665 | | |

References
Acker T, Beck H, Plate K H (2001) Cell type specific expression of vascular endothelial growth factor and angiopoietin-1 and -2 suggests an important role of astrocytes in cerebellar vascularization. Mech Dev 108:45-57.
Angelucci F, Aloe L, Jimenez-Vasquez P, Mathe A A (2002) Electroconvulsive stimuli alter the regional concentrations of nerve growth factor, brain-derived neurotrophic factor, and glial cell line-derived neurotrophic factor in adult rat brain. J ECT 18:138-143.
Bolwig T G, Woldbye D P, Mikkelsen J D (1999) Electroconvulsive therapy as an anticonvulsant: a possible role of neuropeptide Y(NPY). J ECT 15:93-101.
Bonthius D J, Karacay B, Dai D, Pantazis N J (2003) FGF-2, NGF and IGF-1, but not BDNF, utilize a nitric oxide pathway to signal neurotrophic and neuroprotective effects against alcohol toxicity in cerebellar granule cell cultures. Brain Res Dev Brain Res 140:15-28.
Chang C, Werb Z (2001) The many faces of metalloproteases: cell growth, invasion, angiogenesis and metastasis. Trends Cell Biol 11:S37-S43.
Chen A C, Shin K H, Duman R S, Sanacora G (2001a) ECS-induced mossy fiber sprouting and BDNF expression are attenuated by ketamine pretreatment. J ECT 17:27-32.
Chen A C, Eisch A J, Sakai N, Takahashi M, Nestler E J, Duman R S (2001b) Regulation of GFRalpha-1 and GFRalpha-2 mRNAs in rat brain by electroconvulsive seizure. Synapse 39:42-50.
Chen B, Dowlatshahi D, MacQueen G M, Wang J F, Young L T (2001) Increased hippocampal BDNF immunoreactivity in subjects treated with antidepressant medication. Biol Psychiatry 50:260-265.
Chen C, Magee J C, Bazan N G (2002) Cyclooxygenase-2 regulates prostaglandin E2 signaling in hippocampal long-term synaptic plasticity. J Neurophysiol 87:2851-2857.
Chinery R, Beauchamp R D, Shyr Y, Kirkland S C, Coffey R J, Morrow J D (1998) Antioxidants reduce cyclooxygenase-2 expression, prostaglandin production, and proliferation in colorectal cancer cells. Cancer Res 58:2323-2327.
Chodobski A, Szmydynger-Chodobska J (2001) Choroid plexus: target for polypeptides and site of their synthesis. Microsc Res Tech 52:65-82.
Cotter D, Mackay D, Landau S, Kerwin R, Everall I (2001) Reduced glial cell density and neuronal size in the anterior cingulate cortex in major depressive disorder. Arch Gen Psychiatry 58:545-553.
Czeh B, Michaelis T, Watanabe T, Frahm J, de Biurrun G, van Kampen M, Bartolomucci A, Fuchs E (2001) Stress-induced changes in cerebral metabolites, hippocampal volume, and cell proliferation are prevented by antidepressant treatment with tianeptine. Proc Natl Acad Sci USA 98:12796-12801.
Ding X Z, Tong W G, Adrian T E (2000) Blockade of cyclooxygenase-2 inhibits proliferation and induces apoptosis in human pancreatic cancer cells. Anticancer Res 20:2625-2631.
Duman R S, Charney D S (1999) Cell atrophy and loss in major depression. Biol Psychiatry 45:1083-1084.
Duman R S, Heninger G R, Nestler E J (1997) A molecular and cellular theory of depression. Arch Gen Psychiatry 54:597-606.
Duman R S, Malberg J, Thome J (1999) Neural plasticity to stress and antidepressant treatment. Biol Psychiatry 46:1181-1191.
Eagleson K L, Fairfull L D, Salton S R, Levitt P (2001) Regional differences in neurotrophin availability regulate selective expression of VGF in the developing limbic cortex. J Neurosci 21:9315-9324.
Ekstrand A J, Cao R, Bjomdahl M, Nystrom S, Jonsson-Rylander A C, Hassani H, Hallberg B, Nordlander M, Cao Y (2003) Deletion of neuropeptide Y (NPY) 2 receptor in mice results in blockage of NPY-induced angiogenesis and delayed wound healing. Proc Natl Acad Sci USA 100:6033-6038.
Ende G, Braus D F, Walter S, Weber-Fahr W, Henn F A (2000) The hippocampus in patients treated with electroconvulsive therapy: a proton magnetic resonance spectroscopic imaging study. Arch Gen Psychiatry 57:937-943.
Ferrand-Drake M, Wieloch T (1999) The time-course of DNA fragmentation in the choroid plexus and the CA1 region following transient global ischemia in the rat brain. The effect of intra-ischemic hypothermia. Neuroscience 93:537-549.
Gould E, McEwen B S, Tanapat P, Galea L A, Fuchs E (1997) Neurogenesis in the dentate gyrus of the adult tree shrew is regulated by psychosocial stress and NMDA receptor activation. J Neurosci 17:2492-2498.
Hall M C, Young D A, Waters J G, Rowan A D, Chantry A, Edwards D R, Clark I M (2003) The comparative role of activator protein 1 and Smad factors in the regulation of Timp-1 and MMP-1 gene expression by transforming growth factor-beta 1. J Biol Chem 278:10304-10313.
Hawley R J, Scheibe R J, Wagner J A (1992) NGF induces the expression of the VGF gene through a cAMP response element. J Neurosci 12:2573-2581.
Hellsten J, Wennstrom M, Mohapel P, Ekdahl C T, Bengzon J, Tingstrom A (2002) Electroconvulsive seizures increase hippocampal neurogenesis after chronic corticosterone treatment. Eur J Neurosci 16:283-290.
Husum H, Mikkelsen J D, Hogg S, Mathe A A, Mork A (2000) Involvement of hippocampal neuropeptide Y in mediating the chronic actions of lithium, electroconvulsive stimulation and citalopram. Neuropharmacology 39:1463-1473.
Jin K, Zhu Y, Sun Y, Mao X O, Xie L, Greenberg D A (2002) Vascular endothelial growth factor (VEGF) stimulates neurogenesis in vitro and in vivo. Proc Natl Acad Sci USA 99:11946-11950.
Jones M K, Wang H, Peskar B M, Levin E, Itani R M, Sarfeh I J, Tamawski A S (1999) Inhibition of angiogenesis by nonsteroidal anti-inflammatory drugs: insight into mechanisms and implications for cancer growth and ulcer healing. Nat Med 5:1418-1423.
Kondratyev A, Sahibzada N, Gale K (2001) Electroconvulsive shock exposure prevents neuronal apoptosis after kainic acid-evoked status epilepticus. Brain Res Mol Brain Res 91:1-13.
Kondratyev A, Ved R, Gale K (2002) The effects of repeated minimal electroconvulsive shock exposure on levels of mRNA encoding fibroblast growth factor-2 and nerve growth factor in limbic regions. Neuroscience 114:411-416.
Lamont S R, Paulls A, Stewart C A (2001) Repeated electroconvulsive stimulation, but not antidepressant drugs, induces mossy fibre sprouting in the rat hippocampus. Brain Res 893:53-58.
Leahy K M, Ornberg R L, Wang Y, Zweifel B S, Koki A T, Masferrer J L (2002) Cyclooxygenase-2 inhibition by celecoxib reduces proliferation and induces apoptosis in angiogenic endothelial cells in vivo. Cancer Res 62:625-631.
Lee E W, Grant D S, Movafagh S, Zukowska Z (2003) Impaired angiogenesis in neuropeptide Y(NPY)-Y2 receptor knockout mice. Peptides 24:99-106.
Lombardo A, Rabacchi S A, Cremisi F, Pizzorusso T, Cenni M C, Possenti R, Barsacchi G, Maffei L (1995) A developmentally regulated nerve growth factor-induced gene, VGF, is expressed in geniculocortical afferents during synaptogenesis. Neuroscience 65:997-1008.
Louissaint Jr A, Rao S, Leventhal C, Goldman S A (2002) Coordinated interaction ofneurogenesis and angiogenesis in the adult songbird brain. Neuron 34:945-960.
Madsen T M, Greisen M H, Nielsen S M, Bolwig T G, Mikkelsen J D (2000a) Electroconvulsive stimuli enhance both neuropeptide Y receptor Y1 and Y2 messenger RNA expression and levels of binding in the rat hippocampus. Neuroscience 98:33-39.
Madsen T M, Treschow A, Bengzon J, Bolwig T G, Lindvall O, Tingstrom A (2000b) Increased neurogenesis in a model of electroconvulsive therapy. Biol Psychiatry 47:1043-1049.
Madsen T M, Newton S S, Eaton M E, Russell D S, Duman R S (2003) Chronic electroconvulsive seizure up-regulates beta-catenin expression in rat hippocampus: role in adult neurogenesis. Biol Psychiatry 54:1006-1014.
Majima M, Hayashi I, Muramatsu M, Katada J, Yamashina S, Katori M (2000) Cyclo-oxygenase-2 enhances basic fibroblast growth factor-induced angiogenesis through induction of vascular endothelial growth factor in rat sponge implants. Br J Pharmacol 130:641-649.
Malberg J E, Eisch A J, Nestler E J, Duman R S (2000) Chronic antidepressant treatment increases neurogenesis in adult rat hippocampus. J Neurosci 20:9104-9110.
Mallei A, Shi B, Mocchetti I (2002) Antidepressant treatments induce the expression of basic fibroblast growth factor in cortical and hippocampal neurons. Mol Pharmacol 61:1017-1024.
Manji H K, Duman R S (2001) Impairments of neuroplasticity and cellular resilience in severe mood disorders: implications for the development of novel therapeutics. Psychopharmacol Bull 35:5-49.
Matsuoka N, Nozaki K, Takagi Y, Nishimura M, Hayashi J, Miyatake S, Hashimoto N (2003) Adenovirus-mediated gene transfer of fibroblastgrowth factor-2 increases BrdU-positive cells after forebrain ischemia in gerbils. Stroke 34:1519-1525.
Mennicken F, Chabot J G, Quirion R (2002) Systemic administration of kainic acid in adult rat stimulates expression of the chemokine receptor CCR5 in the forebrain. Glia 37:124-138.
Merritt G, Aliprandis E T, Prada F, Rigas B, Kashfi K (2001) The retinoid fenretinide inhibits proliferation and downregulates cyclooxygenase-2 gene expression in human colon adenocarcinoma cell lines. Cancer Lett 164:15-23.
Mimics K, Middleton F A, Marquez A, Lewis D A, Levitt P (2000) Molecular characterization of schizophrenia viewed by microarray analysis of gene expression in prefrontal cortex. Neuron 28:53-67.
Naeve G S, Ramakrishnan M, Kramer R, Hevroni D, Citri Y, Theill L E (1997) Neuritin: a gene induced by neural activity and neurotrophins that promotes neuritogenesis. Proc Natl Acad Sci USA 94:2648-2653.
Nakayama M, Uchimura K, Zhu R L, Nagayama T, Rose M E, Stetler R A, Isakson P C, Chen J, Graham S H (1998) Cyclooxygenase-2 inhibition prevents delayed death of CA1 hippocampal neurons following global ischemia. Proc Natl Acad Sci USA 95:10954-10959.
Nestler E J, Barrot M, DiLeone R J, Eisch A J, Gold S J, Monteggia L M (2002) Neurobiology of depression. Neuron 34:13-25.
Newton S S, Dow A, Terwilliger R, Duman R (2002) A simplified method for combined immunohistochemistry and in situ hybridization in fresh-frozen, cryocut mouse brain sections. Brain Res Brain Res Protoc 9:214-219.
Nibuya M, Morinobu S, Duman R S (1995) Regulation of BDNF and trkB mRNA in rat brain by chronic electroconvulsive seizure and antidepressant drug treatments. J Neurosci 15:7539-7547.
Nibuya M, Nestler E J, Duman R S (1996) Chronic antidepressant administration increases the expression of cAMP response element binding protein (CREB) in rat hippocampus. J Neurosci 16:2365-2372.
Ongur D, Drevets W C, Price J L (1998) Glial reduction in the subgenual prefrontal cortex in mood disorders. Proc Natl Acad Sci USA 95:13290-13295.
Palmer T D, Willhoite A R, Gage F H (2000) Vascular niche for adult hippocampal neurogenesis. J Comp Neurol 425:479-494.
Raballo R, Rhee J, Lyn-Cook R, Leckman J F, Schwartz M L, Vaccarino F M (2000) Basic fibroblast growth factor (Fgf2) is necessary for cell proliferation and neurogenesis in the developing cerebral cortex. J Neurosci 20:5012-5023.
Rajkowska G, Miguel-Hidalgo J J, Wei J, Dilley G, Pittman S D, Meltzer H Y, Overholser J C, Roth B L, Stockmeier C A (1999) Morphometric evidence for neuronal and glial prefrontal cell pathology in major depression. Biol Psychiatry 45:1085-1098.
Reya T, Duncan A W, Ailles L, Domen J, Scherer D C, Willert K, Hintz L, Nusse R, Weissman I L (2003) A role for Wnt signalling in self-renewal of haematopoietic stem cells. Nature 423:409-414.
Rivera S, Tremblay E, Timsit S, Canals O, Ben-Ari Y, Khrestchatisky M (1997) Tissue inhibitor of metalloproteinases-1 (TIMP-1) is differentially induced in neurons and astrocytes after seizures: evidence for developmental, immediate early gene, and lesion response. J Neurosci 17:4223-4235.
Saarelainen T, Hendolin P, Lucas G, Koponen E, Sairanen M, MacDonald E, Agerman K, Haapasalo A, Nawa H, Aloyz R, Emfors P, Castren E (2003) Activation of the TrkB neurotrophin receptor is induced by antidepres-sant drugs and is required for antidepressant-induced behavioral effects. J Neurosci 23:349-357.
Sasaki T, Kitagawa K, Sugiura S, Omura-Matsuoka E, Tanaka S, Yagita Y, Okano H, Matsumoto M, Hori M (2003) Implication of cyclooxygenase-2 on enhanced proliferation of neural progenitor cells in the adult mouse hippocampus after ischemia. J Neurosci Res 72:461-471.
Sattin A, Pekary A E, Lloyd R L (1994) TRH gene products are implicated in the antidepressant mechanisms of seizures. Ann NY Acad Sci 739:135-153.
Shirayama Y, Chen A C, Nakagawa S, Russell D S, Duman R S (2002) Brain-derived neurotrophic factor produces antidepressant effects in behavioral models of depression. J Neurosci 22:3251-3261.
Siuciak J A, Lewis D R, Wiegand S J, Lindsay R M (1997) Antidepressant-like effect of brain-derived neurotrophic factor (BDNF). Pharmacol Biochem Behav 56:131-137.
Snyder S E, Cheng H W, Murray K D, Isackson P J, McNeill T H, Salton S R (1998) The messenger RNA encoding VGF, a neuronal peptide precursor, is rapidly regulated in the rat central nervous system by neuronal activity, seizure and lesion. Neuroscience 82:7-19.
Tan H K, Heywood D, Ralph G S, Bienemann A, Baker A H, Uney J B (2003) Tissue inhibitor of metalloproteinase 1 inhibits excitotoxic cell death in neurons. Mol Cell Neurosci 22:98-106.
Teather L A, Packard M G, Bazan N G (2002) Post-training cyclooxygenase-2 (COX-2) inhibition impairs memory consolidation. Learn Mem 9:41-47.
Thome J, Sakai N, Shin K, Steffen C, Zhang Y J, Impey S, Storm D, Duman R S (2000) cAMP response element-mediated gene transcription is upregulated by chronic antidepressant treatment. J Neurosci 20:4030-4036.
Uchida K, Kumihashi K, Kurosawa S, Kobayashi T, Itoi K, Machida T (2002) Stimulatory effects of prostaglandin E2 on neurogenesis in the dentate gyrus of the adult rat. Zoolog Sci 19:1211-1216.
Vaidya V A, Siuciak J A, Du F, Duman R S (1999) Hippocampal mossy fiber sprouting induced by chronic electroconvulsive seizures. Neuroscience 89:157-166.
van Praag H, Schinder A F, Christie B R, Toni N, Palmer T D, Gage F H (2002) Functional neurogenesis in the adult hippocampus. Nature 415:1030-1034.
Watanabe Y, Gould E, McEwen B S (1992) Stress induces atrophy of apical dendrites of hippocampal CA3 pyramidal neurons. Brain Res 588:341-345.
Wick A, Wick W, Waltenberger J, Weller M, Dichgans J, Schulz J B (2002) Neuroprotection by hypoxic preconditioning requires sequential activation of vascular endothelial growth factor receptor and Akt. J Neurosci 22:6401-6407.
Willert K, Brown J D, Danenberg E, Duncan A W, Weissman I L, Reya T, Yates III J R, Nusse R (2003) Wnt proteins are lipid-modified and can act as stem cell growth factors. Nature 423:448-452.
Yoshimura S, Takagi Y, Harada J, Teramoto T, Thomas S S, Waeber C, Bakowska J C, Breakefield X O, Moskowitz M A (2001) FGF-2 regulation of neurogenesis in adult hippocampus after brain injury. Proc Natl Acad Sci USA 98:5874-5879.
Zhu Y, Jin K, Mao X O, Greenberg D A (2003) Vascular endothelial growth factor promotes proliferation of cortical neuron precursors by regulating E2F expression. FASEB J 17:186-193.
Zonta M, Angulo M C, Gobbo S, Rosengarten B, Hossmann K A, Pozzan T, Carmignoto G (2003) Neuron-to-astrocyte signaling is central to the dynamic control of brain microcirculation. Nat Neurosci 6:43-50.
   Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

Those skilled in the art will know or be able to ascertain using no more than routine experimentation, many equivalents to the embodiments and practices described herein. Accordingly, it will be understood that the invention is not to be limited to the embodiments disclosed herein, but is to be understood from the following claims, which are to be interpreted as broadly as allowed under the law.

1. A gene chip having a plurality of different oligonucleotides attached to a first surface of the solid support and having specificity for genes associated with at least one psychiatric condition.

2. A gene chip according to claim 1, having an oligonucleotide specific for BDNF.

3. A gene chip according to claim 1, having oligonucleotides specific for the genes set out on Table 1, Table 2, Table 3, or a combinations thereof.

4. A gene chip according to claim 1, wherein at least 50% of the oligonucleotides are specific for the genes set out on Table 1, Table 2 or Table 3.

5. A gene chip according to claim 1, having oligonucleotides specific for the genes associated with depression.

6. A gene chip according to claim 1, wherein the psychiatric condition is selected from the group consisting of autism, autism spectrum disorders, Parkinson's disease, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia and related psychiatric disorders, delirium, Tourette's syndrome, myasthenia gravis, attention deficit hyperactivity disorder, dyslexia, mania, depression, apathy, myopathy, Alzheimer's disease, Huntington's Disease, dementia, schizophrenia, severe clinical depression, brain injury, Attention Deficit Disorder (ADD), Attention Deficit Hyperactivity Disorder (ADHD), hyperactivity disorder and Asperger's Disorder.

7. A method for determining a gene signature for a psychiatric condition, comprising
(i) preparing samples of control and experimental cDNA, wherein the experimental cDNA is generated from a nucleic acid sample isolated from a subject afflicted with the psychiatric condition;
(ii) preparing one or more microarrays comprising a plurality of different oligonucleotides attached to a first surface and having specificity for genes associated with the psychiatric condition;
(iii) applying the prepared samples to the one or more microarrays to allow hybridization between the oligonucleotides and the control and experimental cDNAs;
(v) identifying the oligonucleotides on the microarray which display differential hybridization to the experimental cDNA relative to the control cDNA;
(vi) identifying a set of genes from the oligonucleotides identified in step (v),
thereby determining a gene signature for the psychiatric condition.

8. A method according to claim 7, wherein the oligonucleotides comprise those listed in Table 1, 2 or 3, or combinations thereof.

9. A method according to claim 7, wherein at least 50% of the oligonucleotides comprise the genes listed in Tables 1, 2 or 3, or combinations thereof.

10. A method according to claim 7, wherein applying the prepared samples includes controlling the humidity of the environment around the microarray to contain spot size.

11. The method of claim 7, wherein the experimental sample is isolated from a subject afflicted with one or more neurological conditions, psychiatric conditions, or both.

12. The method of claim 11, wherein the psychiatric condition is selected from the group consisting of autism, autism spectrum disorders, Parkinson's disease, parkinsonism, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, Asperger's Disorder, bipolar manic-depressive disorder, ischemia, alcohol addiction, drug addiction, obsessive compulsive disorders, Pick's disease and Binswanger's disease.

13. The method of claim 12, wherein the psychiatric condition is depression.

14. A method of determining a gene signature indicative of administration of a therapeutic treatment to a subject, the method comprising
(i) preparing samples of control and experimental cDNA, wherein the experimental cDNA is generated from a nucleic acid sample isolated from a subject who has received the therapeutic treatment;
(ii) preparing one or more microarrays comprising a plurality of different oligonucleotides attached to a first surface, wherein the oligonucleotides are specific to genes;
(iii) applying the prepared samples to the one or more microarrays to allow hybridization between the oligonucleotides and the control and experimental cDNAs;
(v) identifying the oligonucleotides on the microarray which display differential hybridization to the experimental cDNA relative to the control cDNA;
(vi) identifying a set of genes from the oligonucleotides identified in step (v),
thereby determining a gene signature for the administration of the therapeutic treatment to the subject.

15. The method of claim 14, wherein the oligonucleotides comprise those listed in Table 1, 2 or 3, or combinations thereof.

16. The method of claim 14, wherein at least 50% of the oligonucleotides comprise the genes listed in Tables 1, 2, 3 or 4, or combinations thereof.

17. The method of claim 14, wherein the therapeutic treatment is intended to treat a psychiatric condition.

18. The method of claim 14, wherein the psychiatric condition is selected from the group consisting of autism, autism spectrum disorders, Parkinson's disease, parkinsonism, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia, delirium, Tourette's syndrome, myasthenia gravis, attention deficit hyperactivity disorder, dyslexia, mania, depression, apathy, myopathy, Alzheimer's disease, Huntington's Disease, dementia, encephalopathy, schizophrenia, severe clinical depression, brain injury, Attention Deficit Disorder (ADD), Attention Deficit Hyperactivity Disorder (ADHD), hyperactivity disorder, Asperger's Disorder, bipolar manic-depressive disorder, ischemia, alcohol addiction, drug addiction, obsessive compulsive disorders, Pick's disease and Binswanger's disease.

19. A method for predicting efficacy of a test compound for altering a behavioral response, comprising:
(i) obtaining a gene signature representative of the gene expression profile of at least one sample of a selected tissue type from at least one animal subjected to each of at least one of a plurality of selected behavioral therapies which promote the behavioral response;
(ii) administering the test compound to at least one test animal; and
(iii) comparing gene expression profile data in at least one sample of the selected tissue type from the animal treated with the test compound to determine a degree of similarity with one or more gene signatures;
wherein the predicted efficacy of the test compound for altering the behavioral response is correlated to said degree of similarity.

20. The method of claim 19, wherein step (i) comprises obtaining a gene signature representative of the gene expression profile of at least two samples of a selected tissue type.

21. The method of claim 20, wherein step (i) comprises obtaining a gene signature representative of the gene expression profile of at least three samples of a selected tissue type.

22. The method of claim 19, wherein the selected tissue type comprises a neuronal tissue type.

23. The method of claim 22, wherein the neuronal tissue type is selected from the group consisting of hypothalamus, amygdala, pituitary, nervous system, brainstem, cerebellum, cortex, frontal cortex, hippocampus, striatum, and thalamus.

24. The method of claim 19, wherein the selected tissue type is selected from the group consisting of brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue.

25. The method of claim 19, wherein the test compound is an antibody, a nucleic acid or a small molecule drug.

26. The method of claim 19, wherein the animal is a mammal, a primate, a rodent, a mouse, a rat, a guinea pig, a rabbit or a human.

27. The method of claim 19, wherein the behavioral therapy comprises electroconvulsive seizure therapy, exercise, group therapy, talk therapy, or conditioning.

28. A method of assessing the efficacy of a treatment in an individual having a psychiatric disorder, comprising
(i) determining gene expression profile data in a plurality of patient samples, obtained at multiple time points during treatment of the patient, of a selected tissue type;
(ii) determining a degree of similarity between
(a) the gene expression profile data in the patient samples; and
(b) a gene signature produced by a therapy which has been shown to be efficacious in treatment of the psychiatric disorder;
wherein a high degree of similarity is indicative that the treatment is effective.

29. A kit for identifying a compound for treating a behavioral disorder, comprising
(i) a database having information stored therein gene signature data representative of the genetic expression response of selected tissue type samples from animals that have been subjected to at least one of a plurality of selected behavioral therapies and wherein the tissue has undergone a desired physiological change; and
(ii) a computer program for comparing gene expression profile data obtained from assays wherein a test compound is administered to an animal with the database and providing information representative of a measure of similarity between the gene expression profile data and one or more stored signatures.

30. A method for conducting a drug discovery business, comprising:
(i) generating a database of gene signature data representative of the genetic expression response of a selected neuronal tissue type from an animal that was subjected to at least one of a plurality of behavioral therapies and that has undergone a selected physiological change since commencement of the behavioral therapy;
(ii) selecting at least one gene signature and selecting at least one target as a function of the selected gene signatures;
(iii) screening a plurality of small molecule test agents in assays to obtain gene expression profile data associated with administration of the agents and comparing the obtained data with the one or more selected gene signatures;
(iv) selecting for clinical development test agents that exhibit a desired effect on the target as evidenced by the gene expression profile data;
(v) for test agents selected for clinical development, conducting therapeutic profiling of the test compound, or analogs thereof, for efficacy and toxicity in animals; and
(vi) selecting at least one test agent that has an acceptable therapeutic and/or toxicity profile.

31. The method of claim 30, further including licensing at least one selected test agent to a manufacturer for manufacture and sale of a pharmaceutical preparation comprising said selected agent.

32. The method of claim 30, wherein the behavioral therapy includes electroconvulsive seizure therapy, exercise, group therapy, talk therapy, or conditioning.

33. The method of claim 30, wherein the selected physiological change includes atrophy, growth, loss of neural plasticity, neurogenesis, or sprouting of granule cell mossy fiber pathway.

34. The method of claim 30, wherein, prior to administration of behavioral therapy, the animal showed at least one symptom of a psychological abnormality.

35. The method of claim 30, wherein the animal is afflicted with one or more neurological conditions, a psychiatric condition, or both.

36. The method of claim 35, wherein the psychiatric condition is selected from the group consisting of autism, autism spectrum disorders, Parkinson's disease, cognitive impairments, age-associated memory impairments, cognitive impairments, dementia associated with neurologic and/or psychiatric conditions, epilepsy, brain tumors, brain lesions, multiple sclerosis, Down's syndrome, Rett's syndrome, progressive supranuclear palsy, frontal lobe syndrome, schizophrenia and related psychiatric disorders, delirium, Tourette's syndrome, myasthenia gravis, attention deficit hyperactivity disorder, dyslexia, mania, depression, apathy, myopathy, Alzheimer's disease, Huntington's Disease, dementia, schizophrenia, severe clinical depression, brain injury, Attention Deficit Disorder (ADD), Attention Deficit Hyperactivity Disorder (ADHD), hyperactivity disorder and Asperger's Disorder.

37. The method of claim 30, wherein the neuronal tissue type is selected from the group consisting of hypothalamus, amygdala, pituitary, nervous system, brainstem, cerebellum, cortex, frontal cortex, hippocampus, striatum, and thalamus.

38. A method for conducting a drug discovery business, comprising:
(i) generating a database of gene signature data representative of the genetic expression response of at least one selected neuronal tissue type from an animal that was subjected to at least one of a plurality of behavioral therapies and that has undergone a selected physiological change since commencement of the behavioral therapy;
(ii) administering small molecule test agents to untreated animals to obtain gene expression profile data associated with administration of the agents and comparing the obtained data with the one or more selected gene signatures;
(iii) selecting test agents that induce signatures similar to signatures obtainable by administration of behavioral therapy;
(iv) conducting therapeutic profiling of the selected test compound(s), or analogs thereof, for efficacy and toxicity in animals; and
(v) identifying a pharmaceutical preparation including one or more agents identified in step (v) as having an acceptable therapeutic and/or toxicity profile.

39. The method of claim 38, wherein the neuronal tissue type is selected from the group consisting of hypothalamus, amygdala, pituitary, nervous system, brainstem, cerebellum, cortex, frontal cortex, hippocampus, striatum, and thalamus.

40. The method of claim 38, comprising generating a database of gene signature data representative of the genetic expression response of at least two selected neuronal tissue types.

41. A method for treating a behavioral condition in a patient, comprising identifying a measure of the neural plasticity of a portion of tissue in patient's central nervous system, and administering an agent to the patient in a therapeutic amount sufficient to alter the neural plasticity by a selected amount.
